(19) United States
(12) Patent Application Publication
(10) Pub. No.: US 2024/0397944 A1
(43) Pub. Date: Dec. 5, 2024

(54) METHODS FOR BIOFILM DISRUPTION

(71) Applicant: Neolixir Limited, Nedlands (AU)

(72) Inventors: Angela Fonceca, Osborne Park (AU); Will Ditcham, Hamilton Hill (AU); Gavin Flematti, Leeming (AU); Mark Everard, Derbyshire (GB); Andrew Barker, Melville (AU)

(21) Appl. No.: 18/693,998

(22) PCT Filed: Nov. 11, 2022
(86) PCT No.: PCT/AU2022/051347

§ 371 Date: Mar. 21, 2024

(30) Foreign Application Priority Data
Nov. 12, 2021 (AU) 2021903639

(51) Int. Cl. A01N 37/36; A01P 1/00; A61K 31/19; A61P 31/04

(52) U.S. Cl. CPC A01N 37/36; A01P 1/00; A61K 31/19; A61P 31/04

(57) ABSTRACT

A composition for disrupting a biofilm, said composition comprising a 2-hydroxy carboxylic acid.

### TECHNICAL FIELD

The present invention relates to compositions comprising a hydrophilic 2-hydroxycarboxylic acid for the disruption of bacterial biofilms, and methods of disrupting bacterial biofilms using the aforementioned compositions.

### BACKGROUND ART

Bacterial biofilms are complex surface attached communities of bacteria held together by self-produced polymer matrices. The biofilm matrix consists of substances such as proteins (e.g., fibrin), polysaccharides (e.g., alginate) and extracellular DNA. The dense polysaccharide matrix forms a shield to protect the underlying colony which can be problematic for clearing chronic bacterial infection present across all of nature including human disease, crop maintenance, commercial animal farms, animal disease and biofouling, including marine biofouling, biofouling of membranes, sensors and pipework.

Biofilms are an effective, protective matrix secreted by bacteria. Biofilms encase and sometimes position bacterial colonies, creating an impenetrable barrier to block hostile environments and agents, such as bactericidal or bacteriostatic antibiotics, thereby protecting the bacteria in the biofilm colonies from harm. Further to this, concentration gradients established across the biofilm encased colonies and biofilm matrix are known to induce mechanisms that cause tolerance and/or resistance to antibiotics, and as such biofilm infections are a major contributor to antimicrobial resistance globally. For example, the varied presence of efflux pumps across a biofilm colony can lead to an increased rate of cell mutations resulting in tolerance and/or resistance to an antimicrobial compound. In addition, the presence of extracellular DNA and the close contact between cells in the biofilm matrix favours horizontal gene transfer of tolerant or resistant genes that enable survival. Bacteria in biofilms can be 1000 times more resistant to antimicrobials as a result of these and other mechanisms.

Biofilm formation is detrimental in healthcare, drinking water distribution systems, food, and marine industries, etc. For example, in food industries pathogenic bacteria are able to form biofilms inside of processing facilities, leading to food spoilage and endangering consumer's health. In hospital settings, biofilms have also been shown to persist on medical device surfaces, on patient's tissues or as floating aggregates causing persistent infections and other surfaces such as the inside of soap dispenser pumps. In addition, biofilm forming bacteria contribute to life-threatening infections and diseases in humans or animals such as cystic fibrosis (CF) associated pneumonia, otitis media, periodontitis, sinusitis, infective endocarditis (IE), chronic wounds, ocular infections and osteomyelitis.

The encasing biofilm polysaccharide matrix prevents direct interaction with applied therapeutics, which may be an antibiotic, or agent with a different mode of antimicrobial action, such as a bacteriophage, antiseptic, saline, chlorine-based compound (such as bleach), an iodine-based compound, a copper-based compound, heavy metal etc. used to aid clearance. Ongoing application of antibiotics to biofilm colonies coupled with the ability of these colonies to communicate by quorum sensing has been linked to antibiotic resistance.

The formation of a biofilm can begin with the attachment of free-floating microorganisms to a surface. These first colonists adhere to the surface initially through weak, reversible adhesion via van der Waals forces. If the colonists are not immediately separated from the surface, they can anchor themselves more permanently using cell adhesion structures such as pili. Some species are not able to attach to a surface on their own but are sometimes able to anchor themselves to the matrix or directly to earlier colonists. It is during this colonization that the cells are able to communicate via quorum sensing. Once colonization has begun, the biofilm grows through a combination of cell division and recruitment. Alternatively, the biofilm may begin by the attachment of bacteria to each other, to another organism, or to a non-living particle to form a free-floating biofilm-associated aggregate of bacteria.

Attempts to remove pathogenic bacterial infections with antibiotics can be hampered by the presence of antibiotic resistance which is made more difficult by bacterial biofilms. Failing to successfully remove these bacteria can lead to chronic infection and damage to surrounding mucosal tissues as a result of long-term inflammation.

There is currently a lack of regulated biofilm disruptor therapies available for use. Antibiotics and vaccine therapy remain the gold standard anti-infection treatment approach.

Disrupting, dissolving, removing or otherwise impacting the functionality of the biofilm matrix or colony is an important antimicrobial therapeutic strategy that can be deployed to remove or expose bacterial colonies to the immune system, hostile environments and other antimicrobial agents; or interrupt microbial tolerance and/or resistance mechanisms to antimicrobial treatments. Currently prescribed antibiofilm strategies are limited to mechanical means to remove the biofilm, or chemical treatment with, for example, heavy metals which retard biofilm activity causing their collapse. Novel, scalable, non-toxic antibiofilm therapies are needed to remove or impact problematic biofilm colonies and infections and also reduce resistance to additional antimicrobial therapies across human, animal and plant health; as well as industrial applications such as shipping and wind turbines.

There is a need for alternative treatments to disrupt and/or remove bacterial biofilms; or at least the provision of biofilm disruptors to complement additional bacterial treatments. The present invention seeks to provide an improved or alternative method for bacterial biofilm disruption and removal.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a composition for disrupting a biofilm, said composition comprising a hydrophilic 2-hydroxycarboxylic acid. The hydrophilic 2-hydroxycarboxylic acid preferably has a stereochemistry, and one enantiomer of the stereochemistry is preferred. The 2-hydroxycarboxylic acid of the invention that has the preferred stereochemistry is herein referred to as the “preferred enantiomer of a 2-hydroxycarboxylic acid”, “preferred enantiomer” or “preferred 2-hydroxycarboxylic acid”. In one aspect, the preferred enantiomer of a 2-hydroxycarboxylic acid is D-lactic acid.

Where the preferred 2-hydroxycarboxylic acid is not D-lactic acid, the preferred 2-hydroxycarboxylic acids to be utilised in the present invention may be those in which the absolute stereochemistry of the chiral centre to which the hydroxy group of the 2-hydroxycarboxylic acid corresponds to the absolute stereochemistry of the corresponding chiral centre in D-lactic acid, in terms of the 3-dimensional orientation of the hydroxy group, as depicted below:

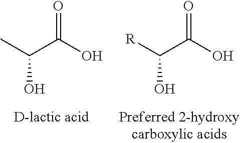

The preferred 2-hydroxycarboxylic acids of the above general formula may be those in which the substituent R is selected from the group consisting of: hydrogen, halogen (F, Cl, Br, I), methyl, ethyl, n-propyl, isopropyl, cyclopropyl, n-butyl, iso-butyl, sec-butyl, tert-butyl, cyclobutyl, n-pentyl, isopentyl, neopentyl, cyclopentyl, n-hexyl, 2-methylpentyl, 3-methylpentyl, 2,3-dimethylbutyl, 2,2-dimethylbutyl, cyclohexyl, n-heptyl, 2-methylhexyl, 3-methylhexyl, 2,3-dimethylpentyl, 2,4-dimethylpentyl, 2,2-dimethylpentyl, 3,3-dimethylpentyl, 3-ethylpentyl, 2,2,3-trimethylbutyl, cycloheptyl, phenyl, benzyl, furanyl, tetrahydrofuranyl, ethenyl, vinyl, allyl, crotyl, isopentenyl, 1-propenyl, 2-propenyl, 1-butenyl, 2-butenyl, 2-methylprop-2-enyl, hexenyl, butadienyl, pentadienyl, hexadienyl, 2,3-dimethyl-2-butenyl, heptenyl, octenyl, octatrienyl, butadienyl, 1,3-pentadienyl, 1,4-pentadienyl, cyclopentadienyl, and cyclohexadienyl, each of which substituents (with the exception of hydrogen and halogen) may be unsubstituted or substituted with one or more substituents selected from the group consisting of: hydrogen, halogen, hydroxyl, methoxyl, ethoxyl, propoxyl, butoxyl, carboxylic acid, amide or ester substituents, and pharmaceutically acceptable salts thereof.

The composition of the present invention may comprise from 0.001% to 100% of total 2-hydroxycarboxylic acid. The rest of the composition may comprise carriers, diluents or excipients, and/or other active agents.

The composition of the present invention may comprise a 2-hydroxycarboxylic acid of an unpreferred stereochemistry, for example L-lactic acid. The 2-hydroxycarboxylic acid of the invention that has the unpreferred stereochemistry is herein referred to as the “unpreferred enantiomer of a 2-hydroxycarboxylic acid”, “unpreferred enantiomer” or “unpreferred 2-hydroxycarboxylic acid”. The % of unpreferred 2-hydroxycarboxylic acid may be less than 20%. Alternatively, the composition of the present invention may not contain an unpreferred 2-hydroxycarboxylic acid. Thus, the composition may comprise only a 2-hydroxycarboxylic acid of a preferred stereochemistry as the 2-hydroxycarboxylic acid.

Where the unpreferred 2-hydroxycarboxylic acid is not L-lactic acid, the unpreferred 2-hydroxycarboxylic acids in accordance with the present invention are those in which the absolute stereochemistry of the chiral centre to which the hydroxy group of the 2-hydroxycarboxylic acid corresponds to the absolute stereochemistry of the corresponding chiral centre in L-lactic acid, in terms of the 3-dimensional orientation of the hydroxy group, as depicted below:

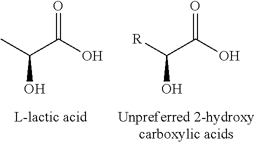

The present invention further provides a method to disrupt a biofilm comprising the step of:
i. administering a preferred enantiomer of a 2-hydroxycarboxylic acid to the biofilm.

The invention further provides a method to disrupt a biofilm comprising the step of:
i. administering a preferred enantiomer of a 2-hydroxycarboxylic acid to the biofilm wherein the preferred enantiomer of a 2-hydroxycarboxylic acid is in combination with an antimicrobial compound.

The present invention further provides a method to treat or prevent a bacterial infection comprising the step of:
i. administering a preferred enantiomer of a 2-hydroxycarboxylic acid to the site of infection.

The invention further provides a method to disrupt or prevent a bacterial infection comprising the step of:
i. administering a preferred enantiomer of a 2-hydroxycarboxylic acid to the site of infection wherein the preferred enantiomer of a 2-hydroxycarboxylic acid is in combination with an antimicrobial compound.

The antimicrobial compound may be an antibiotic, or agent with a different mode of antimicrobial action, such as a bacteriophage, antiseptic, saline, chlorine-based compound (such as bleach), an iodine-based compound, a copper-based compound, heavy metal etc.

The preferred enantiomer of a 2-hydroxycarboxylic acid compositions of the present invention may be used to disrupt biofilms on both living and non-living surfaces, as well as free floating biofilm-associated aggregates of bacteria.

The present invention further provides a composition comprising a preferred enantiomer of a 2-hydroxycarboxylic acid for treating or preventing a bacterial infection in a subject, wherein the bacterial infection is in the form of a biofilm and wherein the composition disrupts the biofilm.

The present invention further provides a composition comprising a preferred enantiomer of a 2-hydroxycarboxylic acid for treating or preventing an infection in a subject, in combination with an antimicrobial compound, wherein the infection is associated with a biofilm and wherein the composition disrupts the biofilm.

The present invention further provides a composition comprising a preferred enantiomer of a 2-hydroxycarboxylic acid for treating or preventing microbial colonisation of a surface, wherein the microbial colonisation is associated with a biofilm and wherein the composition disrupts the biofilm.

The present invention further provides a composition comprising a preferred enantiomer of a 2-hydroxycarboxylic acid for treating or preventing microbial colonisation of a surface, in combination with an antimicrobial compound, wherein the microbial colonisation is associated with a biofilm and wherein the composition disrupts the biofilm.

Preferably the infection or colonisation is infection or colonisation by a bacterium.

The present invention provides for the use of a preferred enantiomer of a 2-hydroxycarboxylic acid in the manufacture of a composition for the disruption of a biofilm.

The present invention further provides for the use of a preferred enantiomer of a 2-hydroxycarboxylic acid for the disruption of a biofilm.

The present invention provides for the use of a preferred enantiomer of a 2-hydroxycarboxylic acid in the manufacture of a composition for the disruption of a biofilm in combination with an antimicrobial compound.

The present invention further provides for the use of a preferred enantiomer of a 2-hydroxycarboxylic acid for the disruption of a biofilm in combination with an antimicrobial compound.

The preferred enantiomer of a 2-hydroxycarboxylic acid may be in the same composition as the antimicrobial compound or may be in separate compositions. The antimicrobial compound may be an antibiotic, or agent with a different mode of antimicrobial action, such as a bacteriophage, antiseptic, saline, chlorine-based compound (such as bleach), an iodine-based compound, a copper-based compound, or heavy metal.

The present invention further provides for the use of a preferred enantiomer of a 2-hydroxycarboxylic acid in the manufacture of a composition for treating or preventing an infection in a subject wherein the infection is in the form of a biofilm and wherein the composition disrupts the biofilm.

The present invention further provides for the use of a preferred enantiomer of a 2-hydroxycarboxylic acid in the manufacture of a composition for treating or preventing an infection in a subject, in combination with an antimicrobial compound, wherein the infection is in the form of a biofilm and wherein the composition disrupts the biofilm.

The present invention further provides for the use of a preferred enantiomer of a 2-hydroxycarboxylic acid for treating or preventing an infection in a subject wherein the infection is in the form of a biofilm and wherein the composition disrupts the biofilm.

The present invention further provides for the use of a preferred enantiomer of a 2-hydroxycarboxylic acid for treating or preventing an infection in a subject, in combination with an antimicrobial compound wherein the infection is in the form of a biofilm and wherein the composition disrupts the biofilm.

The present invention further provides for the use of a preferred enantiomer of a 2-hydroxycarboxylic acid in the manufacture of a composition for treating or preventing microbial colonisation of a surface wherein the microbial colonisation is in the form of a biofilm and wherein the composition disrupts the biofilm.

The present invention further provides for the use of a preferred enantiomer of a 2-hydroxycarboxylic acid in the manufacture of a composition for treating or preventing microbial colonisation of a surface, in combination with an antimicrobial compound, wherein the microbial colonisation is in the form of a biofilm and wherein the composition disrupts the biofilm.

The present invention further provides for the use of a preferred enantiomer of a 2-hydroxycarboxylic acid for treating or preventing microbial colonisation of a surface wherein the microbial colonisation is in the form of a biofilm and wherein the composition disrupts the biofilm.

The present invention further provides for the use of a preferred enantiomer of a 2-hydroxycarboxylic acid for treating or preventing microbial colonisation of a surface, in combination with an antimicrobial compound wherein the microbial colonisation is in the form of a biofilm and wherein the composition disrupts the biofilm.

The present invention provides a kit for the disruption of a biofilm comprising:
a) a preferred enantiomer of a 2-hydroxycarboxylic acid; and
b) instructions for use.

The present invention provides a kit for the disruption of a biofilm comprising:
a) a preferred enantiomer of a 2-hydroxycarboxylic acid; and
b) instructions for use
   wherein the preferred enantiomer of a 2-hydroxycarboxylic acid is in combination with an antimicrobial compound.

In the embodiments above, the preferred enantiomer of a 2-hydroxycarboxylic acid may be D-lactic acid.

In the embodiments above, the preferably the infection or colonisation is infection or colonisation by a bacterium.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIGS. 1A-B are graphs of crystal violet absorbance readings for biofilm-protected microcolonies treated with dilutions of: FIG. 1A-prebiotic test solution (dilutions); and FIG. 1B-tobramycin dose range (ug/ml) (absorbance readings at 600 nm).

FIGS. 2A-B are graphs of the resazurin fluorescence of supernatants collected from biofilm-protected Pseudomonas aeruginosa microcolonies (WACC91) treated with (a) prebiotic test solution and (b) tobramycin. P. aeruginosa cultures were incubated overnight at 37° C. to establish biofilm formation. These were then treated for 24 hrs with dilutions of prebiotic test solution or tobramycin (see Table 2). Following treatment, supernatants were removed and resazurin added to measure metabolic activity of P. aeruginosa released from biofilm. Reduced metabolic activity was observed up to 1 in 24 dilution of the prebiotic test solution and from 1-16 ug/ml tobramycin.

FIG. 3 shows the identification of D-Lactic acid as the active biofilm disrupting agent in the prebiotic test solution. D-Lactic acid was identified following successive testing of HPLC derived fractions to narrow down to a single peak that could be identified by mass spectrometry and nuclear magnetic resonance. Following lactic acid identification, optical rotation showed 95% of the isolated fraction was the D-isomer.

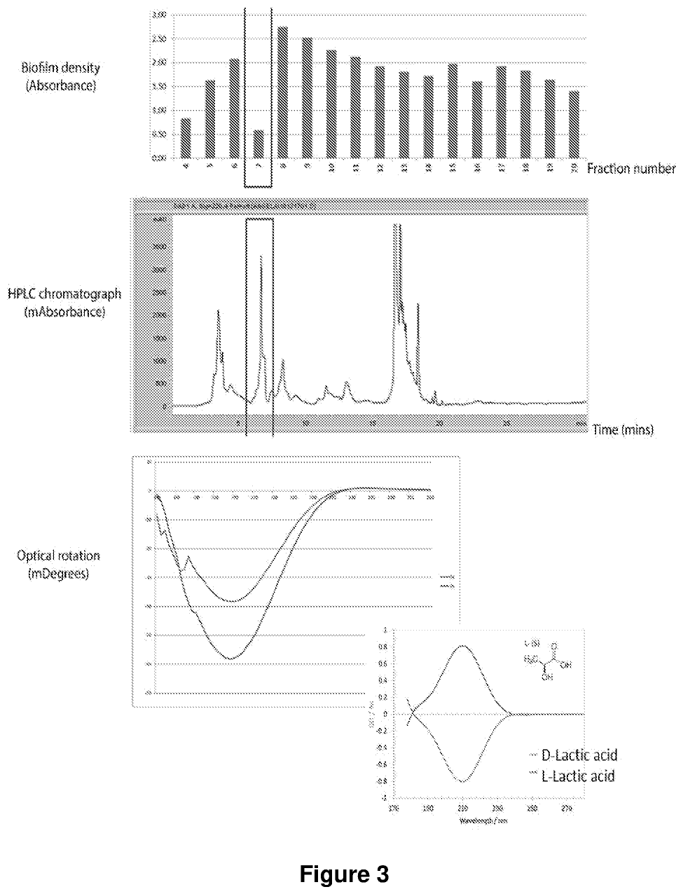

FIG. 4 shows the impact of D-Lactic acid on biofilm-protected microcolonies characterised by biofilm density (green) and viability stains (red indicates death) by confocal microscopy. D-Lactic acid treatment; reduces the size of biofilm-protected microcolonies; disperses biofilm-protected microcolonies, and/or enhances the efficacy of tobramycin.

FIG. 5 is a graph of the biofilm disruption caused by lactic acid enantiomers against Pseudomonas aeruginosa, Staphylococcus aureus and Staphylococcus epidermidis. The lactic acid enantiomers were added at either time zero (0 h) or after 24 h once the biofilm had matured.

FIG. 6 is an image of biofilm microcolony dispersion by addition of D-lactic acid. Single microcolonies of P. aeruginosa WACC91, grown overnight in the 3D biofilm model then either untreated or treated with 1 mg/mL D-Lactic acid or 1 mg/mL L-Lactic acid for 4 h were evaluated with confocal microscopy performed with ‘live/dead’ staining.

### DESCRIPTION OF INVENTION

### Detailed Description of the Invention

Lactic acid is known to be bactericidal due to its ability to reduce the environmental pH and by disrupting the integrity of the cell membrane. These antibacterial properties have been used in food production.

It has further been proposed that lactic acid bacterial biofilms can be used to develop protective probiotic biofilms that exclude potentially pathogenic or contaminating bacteria. The co-aggregation abilities of lactic acid bacterial strains are thought to interfere with the ability of the pathogenic species to infect the host and can prevent the colonization of food-borne pathogens. This method is based on prevention via exclusion of the formation of biofilms by unwanted bacteria by pre-formation of biofilms by lactic acid bacteria.

However, the present invention has surprisingly found that one enantiomer of 2-hydroxycarboxylic acids, referred to in the present application as the “preferred enantiomer of a 2-hydroxycarboxylic acid”, “preferred enantiomer” or the “preferred 2-hydroxycarboxylic acid”, is effective in disrupting bacterial biofilms. This biofilm disruption effect is not provided by the other enantiomer of the 2-hydroxycarboxylic acid, also known as the “unpreferred enantiomer of a 2-hydroxycarboxylic acid”, “unpreferred enantiomer” or the “unpreferred 2-hydroxycarboxylic acid”. It has been found that in some situations the unpreferred enantiomer is not only not active in disrupting biofilms, but can potentially prevent the preferred enantiomer from having its biofilm disrupting effect.

The biofilm disruption effect is not a bactericidal effect; both enantiomers of 2-hydroxycarboxylic acids such as lactic acid are known to have equal bactericidal effects. In some cases, the bactericidal effects of the two enantiomers is due to their effects on pH. In the present invention, the preferred enantiomer is used at a concentration lower than would be used if the preferred enantiomer was being used for its bactericidal effects.

The present invention has found that the preferred enantiomer of a 2-hydroxycarboxylic acid, including D-lactic acid, is a small water soluble antibiofilm agent that can be used for the purpose of disrupting, dissolving, removing or otherwise impacting existing biofilms, or preventing the formation of new bacterial biofilms. Bacterial biofilm infections and contaminations can be treated by the preferred enantiomer of a 2-hydroxycarboxylic acid alone through its antibiofilm efficacy. Alternatively, the preferred enantiomer of a 2-hydroxycarboxylic acid can be used in combination with other antimicrobial agents or therapies, including antibiotics and non-antibiotic antimicrobial compounds, wherein the antibiofilm activity of the preferred enantiomer of a 2-hydroxycarboxylic acid enhances the bacteriostatic and bactericidal efficacy of the antimicrobial agents or therapies.

### Composition

The present invention provides a composition for disrupting a biofilm, said composition comprising a preferred enantiomer of a 2-hydroxycarboxylic acid, such as D-lactic acid.

Generally, the majority of lactic acid bacteria produce a predominance of L-lactic acid. Without being held to any theory, it is believed that the bacteria present in biofilms have evolved to avoid or denature L-lactic acid, in order to prevent the effects of an organic acid on the matrix of the biofilm. The bacteria are less able to avoid or denature the D-isomer of lactic acid, and thus it is able to have a biofilm degrading effect.

Highly pure L-lactic acid can be produced by a broad range of microorganisms, such as bacteria, fungi, algae and cyanobacteria. On the other hand, most of D-lactic acid-producing microorganisms produce either the racemic mixture or other organic acids, such as acetic acid or succinic acid (Alexandri et al (2019) Food Tech & Biotech 57 (3): 293-304).

The composition of the present invention may comprise from 0.001% to 100% of a preferred enantiomer of a 2-hydroxycarboxylic acid, for example D-lactic acid. The composition may comprise from 0.001%, 0.01%, 0.1%, 1%, 10%, 20% 30%, 40% 50%, 60%, 70% 80% or 90% a preferred enantiomer of a 2-hydroxycarboxylic acid. The rest of the composition may comprise carriers, diluents or excipients, and/or other active agents. For example, ocular compositions may comprise from 0.001% to 1% of a preferred enantiomer of a 2-hydroxycarboxylic acid; washing and soaking, dressings and wipes, topical, inhaled or oral compositions may comprise from 0.2% to 100% of a preferred enantiomer of a 2-hydroxycarboxylic acid.

The composition of the present invention may comprise an unpreferred enantiomer of a 2-hydroxycarboxylic acid, wherein the % of the unpreferred enantiomer of a 2-hydroxycarboxylic acid is less than 50%, 40%, 30%, 20%, 10%, 5% or 1%. More preferably the % of the unpreferred enantiomer of a 2-hydroxycarboxylic acid is less than 20%, 18%, 16%, 14%, 12%, 10%, 9%, 8%, 7%, 6%, 5%, 4%, 3%, 2% or 1%. The % of the unpreferred enantiomer of a 2-hydroxycarboxylic acid may be less than 20%.

Alternatively, the composition of the present invention may not contain any unpreferred enantiomer of a 2-hydroxycarboxylic acid. Thus, the composition may comprise only a preferred enantiomer of a 2-hydroxycarboxylic acid as the 2-hydroxycarboxylic acid. For example, the composition may contain only D-lactic acid as the lactic acid. It has been found that a composition comprising an enantiomerically pure amount of the preferred enantiomer of a 2-hydroxycarboxylic acid has a biofilm disrupting effect, a composition comprising an enantiomerically pure amount of the unpreferred enantiomer of a 2-hydroxycarboxylic acid has little or no biofilm disrupting effect and a composition comprising 1:1 ratio of the preferred enantiomer of a 2-hydroxycarboxylic acid to the unpreferred enantiomer of a 2-hydroxycarboxylic acid has a reduced biofilm disrupting effect compared to administering the pure preferred enantiomer of a 2-hydroxycarboxylic acid or has no biofilm disrupting effect.

The composition preferably comprises an enantiomerically enriched amount of the preferred enantiomer of a 2-hydroxycarboxylic acid. The composition may comprise enantiomerically pure preferred enantiomer of a 2-hydroxycarboxylic acid.

Preferably the concentration of a preferred enantiomer of a 2-hydroxycarboxylic acid used in a composition of the present invention is less than the concentration of the preferred enantiomer of a 2-hydroxycarboxylic acid required to kill bacteria.

Without being held to any theory, it is believed that the present invention requires a 2-hydroxycarboxylic acid with a chiral centre at position 2. It is understood that the chiral centre is at position 2 if the aliphatic chain is at least three carbons long; by this definition glycolic acid [2-hydroxy acetic acid] is not chiral as it is less than three carbons in length.

The 2-hydroxycarboxylic acid may be a hydrophilic 2-hydroxycarboxylic acid. That is, the 2-hydroxycarboxylic acid is preferably able to dissolve in water. Without being held to any theory, it is believed that the water-soluble characteristics of the 2-hydroxycarboxylic acid assist in its penetration of the biofilm and biofilm disrupting activity.

It has been found that generally 2-hydroxycarboxylic acids with seven or less carbons (including the carbons of the hydroxycarboxylic acid moiety) are more water soluble. Thus, preferably the 2-hydroxycarboxylic acid of the present invention comprises seven or fewer carbons. For example, 2-hydroxypropanoic acid (also known as lactic acid) comprises three carbons, 2-hydroxypentanoic acid (also known as 2-hydroxyvaleric acid) comprises five carbons and 2-hydroxycaproic acid (also known as 2-hydroxyhexanoic acid) comprises six carbons. More preferably the 2-hydroxycarboxylic acid of the present invention comprises six or fewer carbons. Most preferably the 2-hydroxycarboxylic acid of the present invention comprises five or fewer carbons.

Preferably the molecular weight of the 2-hydroxycarboxylic acid is less than 800, less than 700, less than 600, less than 500. More preferably the molecular weight of the 2-hydroxycarboxylic acid is less than 200, less than 180, less than 150 or less than 100. For example,

Preferably the absolute water solubility of the 2-hydroxycarboxylic acid is more than 0.5 g/L, 0.75 g/L or 1.0 g/L at a pH of 5 to 10, 9 to 6 or more preferably 6 to 8.

Preferably the absolute water solubility of the 2-hydroxycarboxylic acid is more than 0.5 g/L, 0.75 g/L or 1.0 g/L at a temperature of about 20° C.

The water solubility of the 2-hydroxycarboxylic acid may be alternatively defined by its Log P: that is its Octanol-Water partition co-efficient. Log P is also known as Log K_{ow} or the n-octanol-water partition ratio. The general rule is that Log P: Octanol-Water <1.0=water soluble; Log P: Octanol-Water >1.0=hydrophobic. Preferably the 2-hydroxycarboxylic acid of the present invention has a Log P: Octanol-Water <1.0. Some compounds with a Log P: <1.0 may have more than seven carbons; however, their structure allows for water solubility despite the additional carbons. For example, 2-hydroxy-2-phenylacetic acid (also known as mandelic acid) has eight carbons, but due to its cyclical structure, it has a Log P of 0.67 and is therefore water soluble.

Some 2-hydroxycarboxylic acids with special structures, such as 2-hydroxy-2-phenylacetic acid (also known as mandelic acid) that contains a six-membered ring and eight carbons in total, may still be water soluble and have a Log P: Octanol-Water <1.0. Such 2-hydroxycarboxylic acids are considered to be suitable for the present invention.

The 2-hydroxycarboxylic acid may have substituents, such as hydrogen, halogen, hydroxyl, methoxyl, ethoxyl, propoxyl, butoxyl, carboxylic acid, amide or ester substituents.

The 2-hydroxycarboxylic acid may be a pharmaceutically acceptable 2-hydroxycarboxylic acid. More preferably the 2-hydroxycarboxylic acid is GRAS (“generally recognised as safe”), as defined under sections 201 (s) and 409 of the United States Federal Food, Drug, and Cosmetic Act or equivalent regulations.

The 2-hydroxycarboxylic acid can be selected from acids, or the ester, salt, amide, or other derivatives of the group consisting of lactic acid, glycolic acid (2-hydroxyacetic acid), tartaric acid (2,3-dihydroxysuccinic acid), mandelic acid, 1-hydroxycyclohexane-1-carboxylic acid, 2-hydroxy-2-(tetrahydrofuran-2-yl) acetic acid, 2-hydroxy-2-(2-furanyl) ethanoic acid, 2-hydroxy-2-phenylpropionic acid, 2-hydroxy-2-methylpropionic acid, 2-hydroxy-2-methylbutanoic acid, 2-hydroxybutanoic acid, 2-hydroxypentanoic acid, or mixtures thereof.

The 2-hydroxycarboxylic acid may be chosen from the list comprising: 2-hydroxypropanoic acid (also known as lactic acid), 2-hydroxypentanoic acid (also known as 2-hydroxyvaleric acid), 2-hydroxybutyric acid, 2-hydroxyacetic acid (also known as glycolic acid), 2-hydroxyhexanoic acid (also known as 2-hydroxycaproic acid), and 2-hydroxy-2-phenylacetic acid (also known as mandelic acid).

The 2-hydroxycarboxylic acid may be chosen from the list comprising: D-lactic acid, 2-hydroxypentanoic acid, 2-hydroxybutyric acid, 2-hydroxthexanoic acid, (2)-hydroxyphenylacetic acid and 2-hydroxy-2-phenylacetic acid. Preferred 2-hydroxycarboxylic acids include those in Tables 1 and/or 2.

TABLE 1
Solubility of selected 2-hydroxycarboxylic acids

| | No. | | |
|---|---|---|---|
| Name | Carbons | Mw | cLogP |
| 2-hydroxyacetic acid | 2 | 76.051 | −1.0428 |
| 2-hydroxypropanoic acid | 3 | 90.078 | −0.7338 |
| 2-hydroxy-2-methylpropanoic acid | 4 | 104.105 | −0.4248 |
| 2-hydroxybutanoic acid | 4 | 104.105 | −0.2048 |
| 2,3-dihydroxysuccinic acid | 4 | 150.086 | −3.2196 |
| 2-hydroxy-2-methylbutanoic acid | 5 | 118.132 | 0.1042 |
| 2-hydroxy-3-methylbutanoic acid | 5 | 118.132 | 0.1942 |
| 2-hydroxypentanoic acid | 5 | 118.132 | 0.3242 |
| 2-hydroxy-3,3-dimethylbutanoic acid | 6 | 132.159 | 0.5932 |
| 2-hydroxy-2-methylpentanoic acid | 6 | 132.159 | 0.6332 |
| 2-hydroxy-4-methylpentanoic acid | 6 | 132.159 | 0.7232 |
| 2-hydroxy-3-methylpentanoic acid | 6 | 132.159 | 0.7232 |
| 2-hydroxyhexanoic acid | 6 | 132.159 | 0.8532 |
| 2-(furan-2-yl)-2-hydroxyacetic acid | 6 | 142.11 | −0.3216 |
| 2-hydroxy-2-(tetrahydrofuran- | 6 | 146.142 | −0.72665 |
| 2-yl)acetic acid | | | |
| 1-hydroxycyclohexane-1- | 7 | 144.17 | 0.8882 |
| carboxylic acid | | | |
| 2-hydroxy-2,3,3-trimethylbutanoic acid | 7 | 146.186 | 0.9022 |
| 2-hydroxy-2,4-dimethylpentanoic acid | 7 | 146.186 | 1.0322 |
| 2-hydroxy-2,3-dimethylpentanoic acid | 7 | 146.186 | 1.0322 |
| 2-hydroxy-4,4-dimethylpentanoic acid | 7 | 146.186 | 1.1222 |
| 2-hydroxy-3,3-dimethylpentanoic acid | 7 | 146.186 | 1.1222 |
| 2-hydroxy-3,4-dimethylpentanoic acid | 7 | 146.186 | 1.1222 |
| 2-hydroxy-2-methylhexanoic acid | 7 | 146.186 | 1.1622 |
| 2-hydroxy-5-methylhexanoic acid | 7 | 146.186 | 1.2522 |
| 2-hydroxy-4-methylhexanoic acid | 7 | 146.186 | 1.2522 |
| 2-hydroxy-3-methylhexanoic acid | 7 | 146.186 | 1.2522 |
| 2-hydroxyheptanoic acid | 7 | 146.186 | 1.3822 |
| 2-hydroxy-2-phenylacetic acid | 8 | 152.149 | 0.5024 |
| 2-hydroxy-2-phenylacetic acid | 8 | 152.149 | 0.5024 |
| 2-hydroxy-2-phenylpropanoic acid | 9 | 166.176 | 0.8114 |
| 2-(1-hydroxyethyl)benzoic acid | 9 | 166.176 | 0.856 |

TABLE 2
Solubility of selected 2-hydroxycarboxylic acids

| | | Water solubility* |
|---|---|---|
| 2-hydroxycarboxylic acid | AKA | (Mol/L) |
| 2-Hydroxypropanoic acid | lactic acid | 3.22 |
| 2-Hydroxybutyric acid | | 1.93 |
| 2-Hydroxypentanoic acid | 2-hydroxyvaleric acid | 1.64 |
| 2-Hydroxthexanoic acid | 2-hydroxycaproic acid | 0.27 |
| 2-Hydroxy-2-phenylacetic acid | mandelic acid | 1.14 |

Preferably the 2-hydroxycarboxylic acid has both less than seven carbons and a Log P: Octanol-Water <1.0.

Preferably the 2-hydroxycarboxylic acid of the present invention is lactic acid, which ash two enantiomers. Preferably the preferred enantiomer of a 2-hydroxycarboxylic acid is D-lactic acid, also known as (2R)-2-hydroxypropanoic acid and (R)-lactic acid.

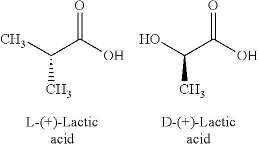

The preferred enantiomer of a 2-hydroxycarboxylic acid may be synthesised via a cell-free route (for example enzyme catalysed reactions), from lactobacillus, from supernatant derived from cultured bacteria such as multi-strain Gram-negative probiotic bacterial strains, or from natural sources such as 2-hydroxy-2-phenylacetic acid (mandelic acid) from almonds, olive oil and beer.

If the preferred enantiomer of a 2-hydroxycarboxylic acid is D-lactic acid, the D-lactic acid may be synthesised via a cell-free route (for example enzyme catalysed reactions), from lactobacillus, or from supernatant derived from cultured bacteria such as multi-strain Gram-negative probiotic bacterial strains.

The D-lactic acid of the present invention is preferably a monomer. Thus, the D-lactic acid is preferably not polylactic acid (also known as PLA, poly D-lactic acid [PDLA], or poly L-lactic acid [PLLA]) or a similar polymer. PLA is a polymer obtained by the ring-opening polymerization of the monomer lactide (cyclic dimer of lactic acid).

The method of the present invention further provides a preferred enantiomer of a 2-hydroxycarboxylic acid to co-administer in combination with an antimicrobial compound, wherein the preferred enantiomer of a 2-hydroxycarboxylic acid increases the activity of the antimicrobial compound against the microorganisms in the biofilm. The preferred enantiomer of a 2-hydroxycarboxylic acid may be in the same composition as the antimicrobial compound, or may be in separate compositions. The antimicrobial compound may be an antibiotic, or agent with a different mode of antimicrobial action, such as a bacteriophage, antiseptic, saline, chlorine-based compound (such as bleach), an iodine-based compound, a copper-based compound, or heavy metal. The biofilm may be an infection of a subject, or may be colonisation of a surface, such as a non-living surface. The biofilm may be a free-floating biofilm-associated aggregate of bacteria.

By “increases the activity”, it is meant that the disruption of the biofilm by the preferred enantiomer of a 2-hydroxycarboxylic acid allows the antimicrobial compound to more easily penetrate to the microorganisms within the biofilm (as the microorganisms are loosened or freed from the matrix of the biofilm) to have its antimicrobial effect.

Preferably, the pH of the compositions of the present invention is from about 6.5 to 8.0, more preferably about 7.0 and 7.4. It has previously been found that bacteria become more resistant to antimicrobial therapy as the pH is lowered. The preferable pH assists in avoiding bacterial resistance to the effect of the preferred enantiomer of a 2-hydroxycarboxylic acid. Buffering agents may be added to adjust the pH level of the composition. Preferably, the compositions of the present invention contain tris (hydroxymethyl) aminomethane (TRIS, also known as THAM or tromethamine) or phosphate-buffered saline (PBS) as a buffering agent. The efficacy of the preferred enantiomer of a 2-hydroxycarboxylic acid at a pH of from about 6.5 to 8.0 is a further indication that the effect on bacterial biofilms is a result of the activity of the 2-hydroxycarboxylic acid itself on the biofilm, not just an effect of low pH killing bacteria.

### Biofilm

The term “biofilm” refers to any syntrophic consortium of microorganisms in which cells stick to each other and often also to a surface; the assemblage of microbial cells, particularly bacterial cells, that is irreversibly associated (not removed by gentle rinsing) with each other and/or the surface. These adherent cells become embedded within a slimy extracellular matrix that is composed of extracellular polymeric substances (EPSs). The cells within the biofilm produce the EPS components, which are typically a polymeric conglomeration of extracellular polysaccharides, proteins, lipids and DNA. The bacterial cells growing in a biofilm are physiologically distinct from planktonic cells (free floating or swimming) of the same organism. A cell, particularly a bacterial cell, that switches to the biofilm mode of growth undergoes a phenotypic shift in behaviour in which large suites of genes are differentially regulated. Biofilms may form on a wide variety of surfaces, including living tissues, indwelling medical devices, industrial or potable water system piping, or natural aquatic systems.

Once a biofilm has been established by the secretion of EPS by bacteria, the dominant microorganisms within the biofilm may shift such that the bacterial load is reduced and other organisms such as fungi and virus predominate. The present invention can still be used to disrupt such biofilms, as the preferred enantiomer of a 2-hydroxycarboxylic acid of the present invention is acting on the biofilm itself, not the microorganisms in the biofilm.

Without being held to any theory, it is believed that the preferred enantiomer of a 2-hydroxycarboxylic acid of the present invention works in three ways. Firstly, the preferred enantiomer of a 2-hydroxycarboxylic acid inhibits the formation of the bacterial biofilm. Secondly, the preferred enantiomer of a 2-hydroxycarboxylic acid disrupts the bacterial biofilm once formed. Thirdly, the preferred enantiomer of a 2-hydroxycarboxylic acid may increase the activity of an antimicrobial compound against the microorganisms in the biofilm. The inhibition and disruption of the biofilm is distinct from the activity of any antimicrobial compound, and may occur in the absence of any further antimicrobial compounds.

When an existing biofilm is disrupted, the microorganisms in the biofilm may be subject to one or more of the following effects:
heightened vulnerability of the microorganisms to host immune system agents, other microbes or other antimicrobial agents including but not limited to antibiotics and bacteriophage;
reduction in the ability of the microorganisms within the biofilm to develop resistance to antimicrobial agents (whether external or from the host);
reduction in growth of the microorganisms within the biofilm;
a reduction in the adherence of the microorganisms the surface on which the biofilm has formed;
a reduction in the rate of formation of the extracellular polymeric substance (EPS) matrix;
a reduction in the viscosity of the EPS matrix.

When inhibition of biofilm formation occurs, the microorganisms in the biofilm may be subject to one or more of the following effects:
reduction in growth of the bacteria that would form the biofilm prior to or during biofilm formation;
heightened vulnerability of the microorganisms to host immune system agents or other antimicrobial agents;
reduction in the ability of the microorganisms within the biofilm to develop resistance to antimicrobial agents (whether external or from the host);
a reduction in the adherence of the bacteria to the surface on which the biofilm will be formed;
a reduction in the rate of formation of the extracellular polymeric substance (EPS) matrix during biofilm formation;
a reduction in the viscosity of the EPS matrix during biofilm formation.

Preferably, the treatment regimens of the present invention cause disruption of biofilm structure wherein biofilm optical density at 600 nm (OD600) is reduced by ≥70%, indicating significant biofilm disruption compared to a control. An example of this measurement is provided in the Examples of the present specification. The disruption of biofilm structure may be determined using crystal violet staining.

Preferably the biofilm is generated by bacteria, however other organisms such as virus, fungi or archaea may also be involved or present in the biofilm. These additional organisms may be predominant in the microbial population of the biofilm.

The bacteria forming the biofilm may be Gram-positive or Gram-negative. For example, the bacteria generating the biofilm may be an Enterobacteriaceae, or may be chosen from the following geniuses: Bacillus spp., Clostridium spp., Campylobacter spp. Pseudomonas spp., Streptococcus spp., Actinobacillus spp., Staphylococcus spp., Escherichia spp., Acinetobacter spp., Klebsiella spp., Aeromonas spp., Enterococcus spp., Legionella spp., and Salmonella spp. Shigella spp., Gardnerella spp., Haemophilus spp., Helicobacter spp., Moraxella spp., Mycobacterium spp., Neisseria spp., Anaerococcus spp., Atopobium spp., Bacteroides spp., Leptotrichia spp., Mobiluncus spp., Peptostreptococcus spp., Prevotella spp., Sneathia spp., Sphingomonas spp., Nitrospira spp., Mycobacterium spp., or Hyphomicrobium spp. and Vibrio spp. The bacteria forming the biofilm may be a cyanobacteria or nontuberculous mycobacteria (NTM).

The bacteria generating the biofilm may be chosen from the bacteria comprising: Pseudomonas aeruginosa, Streptococcus pneumoniae, Streptococcus mutans, Actinobacillus pleuropneumoniae, Staphylococcus epidermidis, Escherichia coli, Staphylococcus aureus, Acinetobacter baumannii, Aeromonas hydrophila, Aeromonas salmonicida, Clostridium difficile, Enterococcus faecium, Gardnerella vaginalis, Haemophilus influenzae, Helicobacter pylori, Moraxella bovis, Moraxella catarrhalis, Mycobacterium avium, Mycobacterium intracellulare, Mycobacterium tuberculosis, Neisseria gonorrhoeae, Neisseria meningitidis, Streptococcus pyogenes, and Vibrio cholerae.

For example, Bacillus spp., Clostridium sp., and Pseudomonas spp. are often found in gas and oil pipelines. Sphingomonas spp., Nitrospira spp., Legionella spp., Mycobacterium spp., or Hyphomicrobium spp. are often found colonising water pipes and air conditioning units. Eubacteria, including cyanobacteria, may form the basis of biofilm fouling on the hulls of ships.

Biofilm formation in bacterial vaginosis caused by or associated with Gardnerella vaginalis and other taxonomic groups such as Anaerococcus, Atopobium, Bacteroides, Leptotrichia, Mobiluncus, Peptostreptococcus, Prevotella, Sneathia and Clostridia spp. may be treated with the biofilm disrupting compositions of the present invention.

The bacteria generating the biofilm may be antibiotic resistant strains of the bacteria listed above. For example, the bacteria generating the biofilm may be antibiotic resistant Pseudomonas aeruginosa or antibiotic resistant Staphylococcus aureus (e.g. MRSA).

The bacteria generating the biofilm may be associated with a condition or disease such as cystic fibrosis associated pneumonia, periodontitis, diabetic ulcers, otitis media, periodontitis, sinusitis, infective endocarditis, ocular infections, glue ear, bacterial vaginosis, and osteomyelitis.

Alternatively, the bacteria generating the biofilm may be associated with industrial and non-biological surfaces such as hydroelectric turbines, oil and gas pipelines, air conditioning units, soap dispensers, industrial water systems, ship hulls, etc.

In further examples, the bacterial biofilm may be associated with medical devices and instruments, such as the bags used to store blood donations and indwelling medical devices.

The bacterial biofilms may further be associated with food production and storage. For example, the biofilms to be disrupted may form on food preparation surfaces, storage containers, or on the foods itself.

The bacterial biofilms may further be associated with animal housing or materials and equipment associated with animal husbandry.

As used herein, “treating” or “treatment” refers to inhibiting a disease or condition, i.e., arresting or reducing its development or at least one clinical or subclinical symptom thereof. “Treating” or “treatment” further refers to relieving the disease or condition, i.e., causing regression of the disease or condition or at least one of its clinical or subclinical symptoms. The benefit to a subject to be treated is either statistically significant or at least perceptible to the subject and/or the physician. In the context of disrupting biofilms, the term treatment includes: (i) a reduction in matrix density; and/or (ii) bacterial dispersion and reversion to planktonic form.

The term “disruption” refers to one or more of the following: a reduction in the viscosity of the extracellular polymeric substance (EPS) matrix of the biofilm, a reduction in production of the EPS matrix, or a reduction in the adherence of the bacteria to the surface on which the biofilm will be formed. The disruption may be due to one of these factors, or two or three of these factors. The disruption causes or results in the bacteria of the biofilm being dispersed, reverting to planktonic form or smaller aggregates and reducing the bacterial potency for antimicrobial resistance building.

### Method of Disruption

The present invention further provides a method to disrupt a biofilm comprising the step of:
i. administering a preferred enantiomer of a 2-hydroxycarboxylic acid to the biofilm.

The invention further provides a method to disrupt a biofilm comprising the step of:
i. administering a preferred enantiomer of a 2-hydroxycarboxylic acid to the biofilm wherein the preferred 2-hydroxycarboxylic acid is in combination with an antimicrobial compound.

The present invention further provides a method to treat or prevent an infection comprising the step of:
i. administering a preferred enantiomer of a 2-hydroxycarboxylic acid to the site of infection.

The invention further provides a method to or prevent disrupt an infection comprising the step of:
i. administering a preferred enantiomer of a 2-hydroxycarboxylic acid to the site of infection wherein the preferred 2-hydroxycarboxylic acid is in combination with an antimicrobial compound.

The antimicrobial compound may be an antibiotic, or agent with a different mode of antimicrobial action, such as a bacteriophage, antiseptic, saline, chlorine-based compound (such as bleach), an iodine-based compound, a copper-based compound, or heavy metal.

The term “infection” includes microbial growth on both biological and non-biological surfaces and tissues. For example, an infection may be the growth of Pseudomonas in lung tissue or may be the growth of Legionella on water pipes.

The composition of the present invention may comprise from 0.001% to 100% of the preferred enantiomer of a 2-hydroxycarboxylic acid. For example, ocular compositions may comprise from 0.001% to 1% of the preferred enantiomer of a 2-hydroxycarboxylic acid; washing and soaking, dressing and wipe, topical, inhaled or oral compositions may comprise from 0.2% to 100% of the preferred enantiomer of a 2-hydroxycarboxylic acid. The rest of the composition may comprise carriers, diluents or excipients, and/or other active agents. Preferably the preferred enantiomer of a 2-hydroxycarboxylic acid is D-lactic acid.

The composition of the present invention may comprise an unpreferred enantiomer of a 2-hydroxycarboxylic acid, wherein the % of unpreferred enantiomer of a 2-hydroxycarboxylic acid may be less than 20%. Alternatively, the composition of the present invention may not contain an unpreferred enantiomer of a 2-hydroxycarboxylic acid. Thus, the composition may comprise only a preferred enantiomer of a 2-hydroxycarboxylic acid as the 2-hydroxycarboxylic acid.

The present invention further provides a composition comprising a preferred enantiomer of a 2-hydroxycarboxylic acid for treating or preventing an infection in a subject, wherein the infection is associated with a biofilm and wherein the composition disrupts the biofilm.

The present invention further provides a composition comprising a preferred enantiomer of a 2-hydroxycarboxylic acid for treating or preventing an infection in a subject, in combination with an antimicrobial compound, wherein the infection is associated with a biofilm and wherein the composition disrupts the biofilm.

The present invention further provides a composition comprising a preferred enantiomer of a 2-hydroxycarboxylic acid for treating or preventing microbial colonisation of a surface, wherein the microbial colonisation is associated with a biofilm and wherein the composition disrupts the biofilm.

The present invention further provides a composition comprising a preferred enantiomer of a 2-hydroxycarboxylic acid for treating or preventing microbial colonisation of a surface, in combination with an antimicrobial compound, wherein the microbial colonisation is associated with a biofilm and wherein the composition disrupts the biofilm.

The efficacy of the method of treating or preventing an infection in a subject by administering a preferred enantiomer of a 2-hydroxycarboxylic acid may be established by determining outcomes other than the state of the biofilm itself. For example, the efficacy may be determined by an increase in the FEV1/FVC ratio, also called Tiffeneau-Pinelli index, of a subject with obstructive or restrictive lung disease after administration of a preferred enantiomer of a 2-hydroxycarboxylic acid. In another example, the efficacy of treatment using a preferred enantiomer of a 2-hydroxycarboxylic acid against an infection in a mammary gland may be established by a reduction in visible inflammation of the outer surface of the mammary tissue, or a reduction in swelling, heat, pain and/or redness. In either the lung example or the mammary tissue example, a further test may be a microbiological assessment of biological fluids from the infection site (eg sputum, milk) to determine the microbial load. If the preferred enantiomer of a 2-hydroxycarboxylic acid is disrupting the biofilm, the microbial load of the infection site should be reduced.

### Surfaces

The preferred enantiomer of a 2-hydroxycarboxylic acid compositions of the present invention may be used to disrupt biofilms on both living and non-living surfaces.

The biofilms may also include additional pathogenic agents such as viruses, fungi, and archaea which associate in the matrix of the biofilm. Biofilms can adhere to almost any type of surface, such as organic surfaces (living or non-living), plastic, metal, glass, soil particles, wood and food products. The present invention may be used to disrupt biofilms on such surfaces.

Living surfaces that may host biofilms that can be disrupted using the compositions of the present invention include: lung mucosal surfaces (particularly the lungs of subjects with cystic fibrosis or bronchiectasis); chronic wounds (including ulcers such as diabetic ulcers); skin surfaces (particular those with skin dysbiosis, acne, skin itchiness, warts, psoriasis and atopic dermatitis), inner vaginal surfaces (including in bacterial vaginosis and vulvovaginal candidiasis), gastrointestinal surfaces (such as small intestinal bacterial overgrowth, Clostridium difficile infections, stomach ulcers), the middle ear surfaces (particularly those with otitis media); periodontic surfaces (particularly those with periodontitis); sinus surfaces (particularly those infected with sinusitis); heart surfaces (particularly those with infective endocarditis), ocular surfaces (particularly those with postoperative endophthalmitis, microbial keratitis, infection crystalline keratopathy), other ocular surfaces (particularly those associated with scleral buckle insertion, conjunctival plug insertion, and lacrimal intubation) and bone surfaces (particularly those with osteomyelitis).

The presence of biofilms is common in food industry and represents a concern because pathogenic bacteria may be involved in the biofilms.

Medical non-living surfaces that may host biofilms that can be disrupted using the compositions of the present invention include indwelling medical devices such as urinary catheters; venous catheters; intrauterine devices; prostheses (including artificial hips, knees, heart valves, etc; cochlear implants; intraocular lenses; breast implants; etc.); vascular access devices; endotracheal tubes; tracheostomies, enteral feeding tubes; wound drains; ear ventilation tubes; soap dispensers and surface medical devices such as contact lenses, orthodontic retainers and mouth guards.

Industrial surfaces that may host biofilms include hydroelectric turbines, oil and gas pipelines, air conditioning units, soap dispensers, industrial water systems, ship hulls, dairy equipment etc. The bacterial biofilms may form on animal housing or materials and equipment associated with animal husbandry.

### Delivery

The compositions of the present invention may be provided as, for instance: topical compositions such as ointments, creams, foams, adhesives or lotions, eye ointments, and eye or ear drops, or nasal sprays; impregnated dressings, patches and wipes; internal local compositions such as pessaries and suppositories; as inhaled compositions such as nebulised or dry powder compositions; as injectable formulations for intravenous and localised injections (for example into an abcess), or as oral formulations in tablet, liquid capsule, oral gel, etc form. The compositions may further be in the form of washes or soaking solutions.

The term a “therapeutically effective amount” as used herein means an amount of the composition, which when administered according to a desired dosage regimen or application regimen, is sufficient to at least partially attain the desired effect, or delay the onset of, or inhibit the progression of, halt, partially or fully the onset or progression of the biofilm.

The term a “preventative effective amount” as used herein means an amount of the composition, which when administered according to a desired dosage regimen or application regimen, is sufficient to at least partially prevent or delay the onset of the biofilm.

Based on the above, it will be understood by those skilled in the art that a plurality of different treatments and means of administration can be used to treat a single subject or surface. For example, subjects already receiving medications, for example as intravenous antibiotics, etc., may benefit from oral delivery, inhalation or topical application of the compositions of the present invention. Some subjects may receive only the present compositions comprising a preferred enantiomer of a 2-hydroxycarboxylic acid by oral administration, inhalation or topical application. For example, subjects may have symptoms of cystic fibrosis, be diagnosed as having lung infections, or have symptoms of a medical condition, which symptoms may benefit from administration to the subject of an inhaled composition comprising a preferred enantiomer of a 2-hydroxycarboxylic acid a preferred 2-hydroxycarboxylic acid. Alternatively, the subject may have a topical infection such as a chronic wound, ocular infection or periodontitis and the preferred enantiomer of a 2-hydroxycarboxylic acid may be administered topically. The subject may have disruptions (“dysbiosis”) within the gut, for example the small intestine, that may benefit from oral administration of the preferred enantiomer of a 2-hydroxycarboxylic acid compositions of the present invention. These dysbioses may contribute to enteropathogen-induced disorders, inflammatory bowel disease (IBS), Crohn's disease, ulcerative colitis or colorectal cancer.

The compositions of the present invention may be used on non-living surfaces, such as in in the form of moist wipes, as washes or soaking solutions for non-living surfaces such as indwelling devices and water pipes, and as cleaning compositions for pipes, food preparation machines and medical equipment. If the composition is being applied to a non-living surface, the D-lactic acid wipe, wash or soak may be followed by washing with conventional detergents, UV sterilization or bleach.

The compositions of the invention may also be used diagnostically. In an embodiment, for example, a subject may receive a dose of a composition of the invention as part of a procedure to diagnose infections associated with biofilms (such as lung infections), wherein one of more of the subject's symptoms improves in response to the composition.

In accordance with certain embodiments, the composition is administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regimen is administered to the subject between 1 and 5 times per day, more preferably once or twice per day.

If the administration is to a non-living surface, the composition may be applied to the surface regularly until removal or disruption of the biofilm is obtained. In one preferred embodiment, the composition is applied to the surface every hour, every 2 hours, every 3 hours, once daily, twice daily, three times daily, four times daily, five times daily, once weekly, twice weekly, once fortnightly and once monthly. However, other application schedules may be utilized in accordance with the present invention. The compositions may be applied to an indwelling device before insertion, and after removal.

Preferably the compositions are administered, for example, orally, topically (ophthalmically, buccally and sublingually, rectally, vaginally, intranasally) or by aerosol administration. If the compositions are to be delivered to non-living surfaces, the compositions may be administered as a wash, soaking solution or wipe. The mode of administration or application is preferably suitable for the form in which the composition has been prepared. The mode of administration for the most effective response may be determined empirically and the means of administration or application described below are given as examples, and do not limit the method of delivery of the composition of the present invention in any way.

The compositions of the invention may optionally include pharmaceutically acceptable nontoxic excipients and carriers. As used herein, a “pharmaceutical carrier” is a pharmaceutically acceptable solvent, suspending agent, excipient or vehicle for delivering the compounds to the subject. The carrier may be liquid or solid and is selected with the planned manner of administration in mind.

The composition of the invention may be selected from the group consisting of: an immediate release composition, a delayed release composition, a controlled release composition and a rapid release composition.

The composition of the invention may further comprise an anti-inflammatory agent (such as a corticosteroid).

The compositions described herein may be formulated by including such dosage forms in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Preferably, the composition contains water, i.e. it is aqueous. In another preferred embodiment, the composition does not comprise a preservative.

Pharmaceutical or veterinary compositions may be formulated according to conventional pharmaceutical or veterinary practices (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18th Edition, Mack Publishing Company, Easton, Pennsylvania, USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the D-lactic acid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

### Washing and Soaking Compositions

The compositions of the present invention comprising a preferred enantiomer of a 2-hydroxycarboxylic acid can be used to wash and/or soak devices such as indwelling medical devices or a water piping systems. Washing or soaking of such devices with the preferred enantiomer of a 2-hydroxycarboxylic acid containing compositions will assist in disrupting any biofilms present on the device surface and thus assist in sterilising or cleaning the device, by rendering the microorganisms present in the biofilm easier to remove and/or kill. The disruption preferably releases the microbes from the encasing biofilm polysaccharide matrix.

In one form, the compositions used for washing or soaking may comprise from 0.1% to 100% of a preferred enantiomer of a 2-hydroxycarboxylic acid. The rest of the composition may comprise carriers, diluents or excipients, and/or other active agents.

In one form, the compositions used for washing or soaking may comprise an unpreferred enantiomer of a 2-hydroxycarboxylic acid, wherein the % of the unpreferred enantiomer of a 2-hydroxycarboxylic acid may be less than 20%. Alternatively, the composition of the present invention may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

### Dressings and Wipes

The compositions of the present invention comprising a preferred enantiomer of a 2-hydroxycarboxylic acid can be provided in the form of impregnated dressings or bandages, or moist wipes. The dressings may be in a form that can be applied to a wound or topical surface that have biofilms, and may be left in situ for a period of hours, days or weeks. Wipes may be used to disrupt biofilms on, for example, wounds or mucosal surfaces such as vaginal or rectal surfaces or on non-living surfaces. The wipes may be used hourly, daily, weekly or as needed. For example, a wipe may be used after each urination or bowel movement to disrupt biofilm formation or disrupt biofilms already present on vaginal or rectal mucosal tissues. Alternatively, a wipe may be used on a food preparation surface prior to use.

In one form, the compositions used in dressings or wipes may comprise from 0.2% to 100% of a preferred enantiomer of a 2-hydroxycarboxylic acid. The rest of the composition may comprise carriers, diluents or excipients, and/or other active agents.

In one form, the compositions used in dressings or wipes may comprise an unpreferred enantiomer of a 2-hydroxycarboxylic acid, wherein the % of the unpreferred enantiomer of a 2-hydroxycarboxylic acid may be less than 20%. Alternatively, the composition of the present invention may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

### Inhaled Delivery

The compositions of the present invention may be delivered via nebulised delivery or via an aerosol device. This is particularly suitable for biofilm related respiratory and ear-nose and throat (ENT) diseases.

Preferably, the composition is administered to the subject in need between about once per day to about six times per day, more preferably about once or twice per day.

Alternatively, the composition may be administered to the subject in need via continuous inhalation, via a nebuliser. The nebulised composition may be delivered for 24 hours, 12 hours, 8 hours, 6 hours, 4 hours, 2 hours or 1 hour, and each of these deliveries (apart from the 24 and 12 hour) may be repeated several times within a 24-hour period.

In one form, the compositions delivered via nebulised or aerosol delivery may comprise from 0.2% to 100% of a preferred enantiomer of a 2-hydroxycarboxylic acid. The rest of the composition may comprise carriers, diluents or excipients, and/or other active agents.

In one form, the compositions delivered via nebulised or aerosol delivery may comprise an unpreferred enantiomer of a 2-hydroxycarboxylic acid, wherein the % of the unpreferred enantiomer of a 2-hydroxycarboxylic acid may be less than 20%. Alternatively, the composition of the present invention may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

A subject may typically receive a dose of from 5 to 500 mg/ml of the preferred enantiomer of a 2-hydroxycarboxylic acid, +20% or +10%. This dose will typically be administered either nebulised or by at least one, preferably several “puffs” from the aerosol device. For example, a subject may receive between 5 mg/ml and 500 mg/ml of the preferred enantiomer of a 2-hydroxycarboxylic acid in a single dose per day, or in several doses over a day.

The total dose per day is preferably administered at least once per day, but may be divided into two or more doses per day. Some subjects may benefit from a period of “loading” the subject with the preferred enantiomer of a 2-hydroxycarboxylic acid with a higher dose or more frequent administration over a period of days or weeks, followed by a reduced or maintenance dose. As cystic fibrosis, COPD etc., are typically chronic conditions, subjects are expected to receive such therapy over a prolonged period of time.

A wide range of mechanical devices designed for pulmonary delivery of therapeutic products exist, including but not limited to nebulizers, metered-dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Missouri; the Acorn Il nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Massachusetts.

All such devices require the use of compositions suitable for the dispensing of the preferred enantiomer of a 2-hydroxycarboxylic acid. Typically, each composition is specific to the type of device employed and may involve the use of an appropriate propellant material, in addition to the usual diluents, adjuvants and/or carriers useful in therapy.

Regardless of the form of the drug composition, it is preferable to create droplets or particles for inhalation in the range of about 0.1 μm to 12 μm, or about 0.25 μm to 6 μm, preferably 1 μm to 6 μm, and more preferably about 2 μm to 4 μm. Alternatively, the particles may be 0.1 μm to 1.0 μm, 0.2 μm to 0.9 μm, 0.3 μm to 0.8 μm, 0.4 μm to 0.7 μm, or 0.5 μm. By creating inhaled particles which have a relatively narrow range of size, it is possible to further increase the efficiency of the drug delivery system and improve the repeatability of the dosing. Thus, it is preferable that the particles not only have a size in the range of 0.1 μm to 12 μm or 2 μm to 6 μm or about 3 to 4 μm but that the mean particle size be within a narrow range so that 80% or more of the particles being delivered to a subject have a particle diameter which is within +20% of the average particle size, preferably +10% and more preferably +5% of the average particle size.

“Particle size” is a notion introduced for comparing dimensions of solid particles, liquid particles (droplets). For droplets and aerosols, terms such as “aerodynamic diameter” and “mass median aerodynamic diameter (MMAD) are used. The definitions are given below.

“Aerodynamic diameter” is the diameter of a unit-density sphere having the same terminal settling velocity as the particle in question. It is used to predict where in the respiratory tract such particles will deposit.

“Mass Median Aerodynamic Diameter” is the geometric mean aerodynamic diameter. Fifty percent of the particles by weight will be smaller than the MMAD, 50% will be larger.

During particle sizing experiment, the suspensions contain innumerable number of particles of varying sizes in motion. When the particle-sizing machine analyses these particles, it forms a particle distribution curve, which covers the entire particle size range starting from the smallest particle, which could be 1 nm to the largest, which could be 100 μm. In the particle size distribution curve, a cumulative frequency is calculated for the particles. D₁₀ refers to that particular particle diameter where 10% of the particles in the suspension have a smaller diameter or equal diameter as that of the particular particle diameter.

D₅₀: Similar to the D₁₀, D₅₀ is the cut off diameter for 50% of the particle population in the composition and refers to that particular particle diameter where 50% of the particles in the suspension have a smaller diameter or equal diameter as that of the particular particle diameter.

D₉₀: D₉₀ is the cut off diameter for 90% of the particle population in the composition and refers to that particular particle diameter where 90% of the particles in the suspension have a smaller diameter or equal diameter as that of the particular particle diameter.

The term “respiratory tract” shall be taken to mean a system of cells and organs functioning in respiration, in particular the organs, tissues and cells of the respiratory tract include, lungs, nose, nasal passage, paranasal sinuses, nasopharynx, larynx, trachea, bronchi, bronchioles, respiratory bronchioles, alveolar ducts, alveolar sacs, alveoli, pneumocytes (type 1 and type 2), ciliated mucosal epithelium, mucosal epithelium, squamous epithelial cells, mast cells, goblet cells, and intraepithelial dendritic cells.

In one form of the invention, the method of treating or preventing biofilm development in the lung of a subject comprises administering a therapeutically effective or preventative effective concentration of the preferred enantiomer of a 2-hydroxycarboxylic acid, in the form of one or more doses of from 1 to 1000 mg/ml, more preferably from 5 to 500 mg/ml.

In one form of the invention, the method of treating biofilm development in the lung of a subject comprises administering a therapeutically effective concentration of an inhaled composition comprising a preferred enantiomer of a 2-hydroxycarboxylic acid, in the form of one or more doses of from 1 to 1000 mg/kg/day, more preferably from 6 to 600 mg/kg/day.

In one form of the invention, the method of preventing biofilm development in the lung of a subject by administering a preventative effective concentration of an inhaled composition comprising a preferred enantiomer of a 2-hydroxycarboxylic acid, in the form of one or more doses of from 1 to 1000 mg/ml, more preferably from 5 to 500 mg/ml.

The compositions of the invention may be administered to a subject using a disposable package and portable, hand-held, battery-powered device, such as the AERx device (U.S. Pat. No. 5,823,178, Aradigm, Hayward, Calif.). Alternatively, the compositions of the instant invention may be carried out using a mechanical (non-electronic) device. Other inhalation devices may be used to deliver the compositions including conventional jet nebulizers, ultrasonic nebulizers, soft mist inhalers, dry powder inhalers (DPIs), metered dose inhalers (MDIs), condensation aerosol generators, and other systems.

For use as aerosols, the compounds of the present invention in solution or suspension may be packaged in a pressurized aerosol container together with suitable propellants, for example, hydrocarbon propellants like propane, butane, or isobutane with conventional adjuvants. A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose.

An aerosol may be created by forcing drug through pores of a membrane which pores have a size in the range of about 0.25 to 6 μm (U.S. Pat. No. 5,823,178). When the pores have this size the particles which escape through the pores to create the aerosol will have a diameter in the range of 0.5 to 12 μm. Drug particles may be released with an air flow intended to keep the particles within this size range. The creation of small particles may be facilitated by the use of the vibration device which provides a vibration frequency in the range of about 800 to about 4000 kilohertz. Those skilled in the art will recognize that some adjustments can be made in the parameters such as the size of the pores from which drug is released, vibration frequency, pressure, and other parameters based on the density and viscosity of the composition keeping in mind that an object of some embodiments is to provide aerosolized particles having a diameter in the range of about 0.5 to 12 μm.

### Topical Delivery

The compositions of the present invention may be delivered topically. The topical administration may comprise the administration of the therapeutically effective amount of a preferred enantiomer of a 2-hydroxycarboxylic acid directly to a dermal, ocular or mucosal surface of the subject. Preferably, the preferred enantiomer of a 2-hydroxycarboxylic acid is applied topically to the skin, mucosal membranes (oral, nasal, vaginal, rectal) or eye of the subject. The use may comprise administering a therapeutically effective amount of the preferred enantiomer of a 2-hydroxycarboxylic acid to the skin, mucosal membranes (oral, nasal, vaginal, rectal) or eye of a subject.

Compositions of the invention may be administered topically. Therefore, contemplated for use herein are compositions adapted for the direct application to the skin.

In one form, the compositions used for topical delivery may comprise from 0.2% to 100% of the preferred enantiomer of a 2-hydroxycarboxylic acid. The rest of the composition may comprise carriers, diluents or excipients, and/or other active agents.

In one form, the compositions used for topical delivery may comprise an unpreferred enantiomer of a 2-hydroxycarboxylic acid, wherein the % of the unpreferred enantiomer of a 2-hydroxycarboxylic acid may be less than 20%. Alternatively, the composition of the present invention may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

The composition may be in a form selected from the group comprising suspensions, emulsions, liquids, creams, oils, lotions, ointments, gels, hydrogels, pastes, plasters, roll-on liquids, skin patches, sprays, glass bead dressings, synthetic polymer dressings and solids. For instance, the compositions of the invention may be provided in the form of a water-based composition or ointment which is based on organic solvents such as oils. Alternatively, the compositions of the invention may be applied by way of a liquid spray comprising film forming components and at least a solvent in which the preferred enantiomer of a 2-hydroxycarboxylic acid is dispersed or solubilised.

The composition of the invention may be provided in a form selected from the group comprising, but not limited to, a rinse, a shampoo, a lotion, a gel, a leave-on preparation, a wash-off preparation, and an ointment.

Various topical delivery systems may be appropriate for administering the compositions of the present invention depending up on the preferred treatment regimen. Topical compositions may be produced by dissolving or combining the preferred enantiomer of a 2-hydroxycarboxylic acid of the present invention in an aqueous or non-aqueous carrier. In general, any liquid, cream, or gel or similar substance that does not appreciably react with the compound or any other of the active ingredients that may be introduced into the composition and which is non-irritating is suitable. Appropriate non-sprayable viscous, semi-solid or solid forms can also be employed that include a carrier compatible with topical application and have dynamic viscosity preferably greater than water.

Suitable compositions are well known to those skilled in the art and include, but are not limited to, solutions, suspensions, emulsions, creams, gels, ointments, powders, liniments, salves, aerosols, transdermal patches, etc., which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the preferred 2-hydroxycarboxylic acid is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface-active agent, such as sodium lauryl sulfite; hydrophilic colloids, such as acacia colloidal clays, veegum and the like. Upon formation of the emulsion, the preferred enantiomer of a 2-hydroxycarboxylic acid can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the preferred enantiomer of a 2-hydroxycarboxylic acid is added to the composition at the desired concentration at a point preceding addition of the gelling agent.

Topically delivered compositions for application to mucosal surfaces such as oral, vaginal, nasal or rectal mucosal surfaces, or skin wounds, may comprise from 5% to 100% of the preferred enantiomer of a 2-hydroxycarboxylic acid. The rest of the composition may comprise carriers, diluents or excipients, and/or other active agents. In one aspect. the higher dosages may be used in medically supervised situations, and the lower dosages may be used for non-life-threatening wounds treated at home.

In one form, the compositions used for application to mucosal surfaces and/or skin wounds may comprise an unpreferred enantiomer of a 2-hydroxycarboxylic acid, wherein the % of the unpreferred enantiomer of a 2-hydroxycarboxylic acid may be less than 20%. Alternatively, the composition of the present invention may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

Compositions of the invention may be administered via topical ocular delivery. Preferably, the ocular composition comprises from 0.001% to 1% of the preferred enantiomer of a 2-hydroxycarboxylic acid.

Ocular delivery encompasses delivery to the sclera, retina, intraocular fluid, tissue surrounding the eyeball. For example, the delivery may be topical delivery (creams, gels, ointments, sprays, eye drops), intraocular implant or other means.

Ocular delivery may also comprise injecting the preferred enantiomer of a 2-hydroxycarboxylic acid into the sclera, intraocular space or into the area behind the eye. Compositions suitable for ocular injection optionally include sterile aqueous solutions (where water-soluble) or dispersions and sterile powders for the extemporaneous preparation of sterile injectable solutions or dispersion. Alternatively, the compounds of the invention are, in certain aspects encapsulated in liposomes and delivered in injectable solutions to assist their transport across cell membrane. Alternatively, or in addition, such preparations contain constituents of self-assembling pore structures to facilitate transport across the cellular membrane.

### Orally Delivered Compositions

Compositions of the invention may be administered via oral delivery.

In one form, the compositions delivered orally may comprise from 5% to 100% of the preferred enantiomer of a 2-hydroxycarboxylic acid. The rest of the composition may comprise carriers, diluents or excipients, and/or other active agents.

In one form, the compositions delivered orally may comprise an unpreferred enantiomer of a 2-hydroxycarboxylic acid, wherein the % of the unpreferred enantiomer of a 2-hydroxycarboxylic acid may be less than 20%. Alternatively, the composition of the present invention may not contain any unpreferred enantiomer of a 2-hydroxycarboxylic acid. Thus, the composition may comprise only a preferred enantiomer of a 2-hydroxycarboxylic acid as the 2-hydroxycarboxylic acid.

A subject may typically receive a dose of from 0.1 mg/kg/day to 2 g/kg/day of a preferred enantiomer of a 2-hydroxycarboxylic acid, +20% or +10%.

Despite the varying pH of the digestive system (acidic in the stomach, alkaline in the small intestine), the efficacy of the preferred enantiomer of a 2-hydroxycarboxylic acid against biofilms remains. The pH of the surrounding fluids does not affect the biofilm disrupting capabilities of the preferred enantiomer of a 2-hydroxycarboxylic acid. This is because the 2-hydroxycarboxylic acid remains stable in the gastrointestinal tract and has shown to maintain its functionality, as observed in many studies investigating the acid resistance of lactic acid bacteria (such as Wang et al, 2018, Archives of Microbiology, 200, 195-201) and lactic acid acidosis a metabolic acidosis that occurs from excessive fermentation in the gastrointestinal tract.

The oral compositions of the present invention may be delivered with an immune boosting or modulating agents. Examples of immune boosting or modulating agents include specific immunostimulants (such as vaccines and antigens) and non-specific agents such as honeybee products (including propolis and honey), pro- and pre-biotics, hormones, vitamins (vitamin c, vitamin d), minerals (zinc oxide), antioxidants (including glutathione), interferons (including INF-alpha), interleukins (including interleukin 10), colchicine, thalidomide and imiquimod. The immune booster may be delivered at the same time as the composition comprising a preferred enantiomer of a 2-hydroxycarboxylic acid, or after the composition comprising a preferred enantiomer of a 2-hydroxycarboxylic acid.

### Subject

The subject may be any subject capable of infection by a bacteria, virus, fungi or archaea. The subject may be mammalian or avian. Preferably, the subject is selected from the group comprising human, canine, avian, porcine, bovine, ovine, equine, rodent, mustelid, lagomorph and feline. Most preferably, the subject is selected from the group comprising human, bovine, porcine, equine, feline, rodent, mustelid, lagomorph and canine. The subject may be a companion animal, domestic animal, or animal of agricultural importance. Most preferably, the subject is human.

### Excipients

The above-exemplified forms of the compositions described herein can be manufactured by methods well known to one of skill in the art of composition science. Additionally, the compositions described herein may include other optional excipients to aid in the manufacturing and/or administration of the compositions described herein. In one embodiment, the composition further comprises one or more pharmaceutically or physiologically acceptable carriers, diluents or excipients. Non-limiting examples of such excipients are well known in the art and include flavourings, colorants, palatants, antioxidants, viscosity modifying, tonicity agents, drug carriers, sustained-release agents, comfort-enhancing agents, emulsifiers, solubilizing aids, lubricants, binding agents and other stabilizing agents to aid in the manufacturing and/or administration of the compositions.

Compositions of the invention are intended for pharmaceutical or veterinary use, or for use in cleaning equipment such as medical devices, water pipes or food preparation equipment.

Preferably, the present composition is sterile. In addition to or in lieu of sterilization, the compositions of the present invention may contain a pharmaceutically acceptable preservative to minimize the possibility of microbial contamination. In another embodiment, the composition of the present invention is stable. A pharmaceutically acceptable preservative may be used in the present compositions to increase the stability of the compositions. It should be noted, however, that any preservative must be chosen for safety, as the treated tissues may be sensitive to irritants. Preservatives suitable for use herein include, but are not limited to, those that protect the solution from contamination with pathogens, including phenylethyl alcohol, benzalkonium chloride or benzoic acid, or benzoates such as sodium benzoate and phenylethyl alcohol. In certain embodiments, the compositions herein comprise from about 0.001% to about 10.0% w/w of benzalkonium chloride, or from about 0.01% v/w phenylethyl alcohol. Preserving agents may also be present in an amount from about 0.001% to about 1%, preferably about 0.002% to about 0.02%, more preferably 0.02% w/w.

The compositions provided herein may also comprise from about 0.001% to about 90%, or about 0.001% to about 50%, or about 0.001% to about 25%, or about 0.001% to about 10%, or about 0.001% to about 1% of one or more emulsifying agent, wetting agent, or suspending agent. Such agents for use herein include, but are not limited to, polyoxyethylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan monooleate, polyoxyethylene (20) sorbitan monopalmitate, polyoxyethylene (20) sorbitan monostearate; lecithins; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-21 actylate; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerised fatty acids of soya bean oil; oxidatively polymerised soya bean oil; and pectin extract.

The compositions of the present invention may comprise from about 0.001% to about 5% by weight of a humectant to inhibit drying of the mucous membrane and to prevent irritation. Any of a variety of pharmaceutically acceptable humectants can be employed, including sorbitol, propylene glycol, polyethylene glycol, glycerol or mixtures thereof, for example.

The invention encompasses variations on the above composition, as the amounts of the respective compounds may vary by ±5%, ±7.5%, ±10%, ±15%, ±17.5%, or ±20%.

The present invention encompasses compositions wherein the relative proportions of the active ingredient and/or each excipient independently vary from those specified above. In one form of the invention, the relative proportions of the active ingredient and/or each excipient independently vary by up to 50% from those specified above. In one form of the invention, the relative proportions of the active ingredient and/or each excipient independently vary by up to 40% from those specified above. In one form of the invention, the relative proportions of the active ingredient and/or each excipient independently vary by up to 30% from those specified above. In one form of the invention, the relative proportions of the active ingredient and/or each excipient independently vary by up to 20% from those specified above. In one form of the invention, the relative proportions independently vary by up to 10% from those specified above. In one form of the invention, the relative proportions of the active ingredient and/or each excipient independently vary by up to 5% from those specified above. In one form of the invention, the relative proportions independently vary by up to 10% from those specified above. In one form of the invention, the relative proportions of the active ingredient and/or each excipient independently vary by up to 2% from those specified above.

As would be understood by a person skilled in the art, the sum of the percentages of the excipients and the active cannot exceed 100, and the variations described above are subject to this limitation. As would be understood by a person skilled in the art, the sum of the percentages of the excipients and the active may be less than 100, as forms of the invention include components other than those specified.

The variation described above is a percentage variation of a relative proportion. By way of example, a 20% variation of the relative proportion of a component (excipient or active) that is specified at 1% means that the relative proportion of that component may be 0.8-1.2%.

### Inhaled Delivery Excipients

Compositions suitable for use with a nebulizer, either jet or ultrasonic, will typically comprise the preferred enantiomer of a 2-hydroxycarboxylic acid suspended in water or non-aqueous solvent. The composition may also include a buffer and a simple sugar (e.g., for stabilization and regulation of osmotic pressure). The nebulizer composition may also contain a surfactant, to reduce or prevent surface induced aggregation of the preferred enantiomer of a 2-hydroxycarboxylic acid caused by atomization of the solution in forming the aerosol. Also, the use of liposomes, microcapsules or microspheres, inclusion complexes, or other types of carriers is contemplated.

Compositions for use with a metered dose inhaler device will generally comprise a finely divided powder containing the preferred enantiomer of a 2-hydroxycarboxylic acid suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Compositions for dispensing from a powder inhaler device will comprise a finely divided dry powder containing the preferred enantiomer of a 2-hydroxycarboxylic acid and may also include a bulking agent, such as lactose, sorbitol, sucrose, or mannitol in amounts which facilitate dispersal of the powder from the device, e.g., 50 to 90% by weight of the composition. The preferred enantiomer of a 2-hydroxycarboxylic acid should most advantageously be prepared in particulate form with an average particle size of less than 10 microns, most preferably 0.5 to 5 microns, for most effective delivery to the distal lung.

In one embodiment, the composition of the present invention may comprise a preservative, suspending agent, wetting agent, tonicity agent and/or diluent. The compositions provided herein may comprise from about 0.01% to about 90%, or about 0.01% to about 50%, or about 0.01% to about 25%, or about 0.01% to about 10%, or about 0.01% to about 5% of one or more pharmacologically suitable suspending fluids which is physiologically acceptable upon administration by inhalation. Pharmacologically suitable fluids for use herein include, but are not limited to, polar solvents, including, but not limited to, compounds that contain hydroxyl groups or other polar groups. Solvents include, but are not limited to, water or alcohols, such as ethanol, isopropanol, and glycols including propylene glycol, polyethylene glycol, polypropylene glycol, glycol ether, glycerol and polyoxyethylene alcohols. Polar solvents also include protic solvents, including, but not limited to, water, aqueous saline solutions with one or more pharmaceutically acceptable salt(s), alcohols, glycols or a mixture there of. In one alternative embodiment, the water for use in the present compositions should meet or exceed the applicable regulatory requirements for use in inhaled drugs.

In one embodiment, the compositions described herein may be aqueous and contain 0-90% water. In other embodiments, the aqueous compositions described herein may contain 20-80% water. In still other embodiments, aqueous compositions may contain 50-70% water. The water may further comprise water that is plain, distilled, sterile, demineralized or deionized. Alternatively, the composition may be non-aqueous and contain no water, or negligible amounts of water (e.g. below 1%, below 0.1%, below 0.01%).

The composition of the present invention may further comprise an adjuvant, such as: a bronchodilator, an anti-inflammatory agent, a surfactant, aspirin, or ethyl alcohol.

Bronchodilators optionally used in the compositions of the invention include but are not limited to β2-adrenergic receptor agonists (such as albuterol, bambuterol, salbutamol, salmeterol, formoterol, arformoterol, levosalbutamol, procaterol, indacaterol, carmoterol, milveterol, procaterol, terbutaline, and the like), and antimuscarinics (such as trospium, ipratropium, glycopyrronium, aclidinium, and the like). Combinations of drugs may be used.

Anti-inflammatoires that may optionally be used in the compositions of the invention include but are not limited to inhaled corticosteroids (such as beclometasone, budesonide, ciclesonide, fluticasone, etiprednol, mometasone, and the like), leukotriene receptor antagonists and leukotriene synthesis inhibitors (such as montelukast, zileuton, ibudilast, zafirlukast, pranlukast, amelubant, tipelukast, and the like), cyclooxygenase inhibitors (such as ibuprofen, ketoprofen, ketorolac, indometacin, naproxen, zaltoprofen, lornoxicam, meloxicam, celecoxib, lumiracoxib, etoricoxib, piroxicam, ampiroxicam, cinnoxicam, diclofenac, felbinac, lornoxicam, mesalazine, triflusal, tinoridine, iguratimod, pamicogrel, and the like). Combinations of drugs may be used. Aspirin may also be added to act as an anti-inflammatory agent.

Surfactants covered by the invention include but are not limited to synthetic surfactant (Exosurf®), dipalmitoylphosphatidylcholine and oleic acid. Combinations of drugs may be used. Antioxidants such as glutathione and vitamin E, zinc and zinc salts of EDTA, may be added.

Ethyl alcohol vapour acts as an anti-foaming agent in the lungs and makes sputum more liquid, which can aid breathing and reduce lung oedema. Ethanol may be added to the compositions of the present invention at between 0.5% and 60%, more preferably between 1 and 40%, 1 and 20%, or 1 and 10%. The ethanol may be added at 1%, 2%, 3%, 4%, 5%, 6%, 7%, 8%, 9%, 10%, 15%, 20%, 25%, 30%, 35%, 40%, 45%, 50%, 55% or 60%.

The invention also relates to the use of a preferred enantiomer of a 2-hydroxycarboxylic acid in combination with other drugs given via inhalation. These other drugs may include a nucleotide sequence which may be incorporated into a suitable delivery vector such as a plasmid or viral vector. The other drug may be a therapeutic nucleotide sequence (DNA, RNA, siRNA), enzymes to reduce the viscoelasticity of the mucus such as DNase and other mucolytic agents, chemicals to upregulate the chloride ion channel or increase flow of ions across the cells, nicotine, P2Y2 agonists, elastase inhibitors including α-1 antitrypsin (AAT), N-acetylcysteine, antibiotics and cationic peptides, such as lantibiotics, and specifically duramycin, short-acting bronchodilators (e.g., 32-adrenergic receptor agonists like albuterol or indacaterol), M3 muscarinic antagonists (e.g., ipatropium bromide), K⁺-channel openers, long-acting bronchodilators (e.g., formoterol, salmeterol), steroids (e.g., budesonide, fluticasone, triamcinolone, beclomethasone, ciclesonide, etc.), xanthines, leukotriene antagonists (e.g., montelukast sodium), phosphodiesterase 4 inhibitors, adenosine receptor antagonists, other miscellaneous anti-inflammatoires (e.g., Syk kinase inhibitors (AVE-0950), tryptase inhibitors (AVE-8923 & AVE-5638), tachykinin antagonists (AVE-5883), inducible nitric oxide synthase inhibitors (GW-274150) and others), transcription factor decoys, TLR-9 agonists, antisense oligonucleotides, siRNA, DNA, CGRP, lidocaine, inverse β2-agonists, anti-infective oxidative therapies, cytokine modulators (e.g., CCR3 receptor antagonists (GSK-766994, DPC-168, AZD-3778), TNF-α production inhibitors (LMP-160 & YS-TH2), and IL-4 antagonists (AVE-0309)), small molecule inhibitors of IgE, cell adhesion molecule (CAM) inhibitors, small molecules targeting the VLA4 receptor or integrin.alpha.4.beta.1 (e.g., R-411, PS-460644, DW-908e, & CDP-323), immunomodulators including those that block T-cell signalling by inhibition of calcineurin (Tacrolimus), heparin neutralizers (Talactoferrin a), cytosolic PLA2 inhibitors (Efipladib), or combinations thereof. If the subject in need has CF, then they may also be administered standard medications such as ivacaftor, pulmozyme, mannitol, or other approved drugs according to standard practise, in combination with the compositions of the present invention.

### Topical Delivery Excipients

The compositions of the present invention comprising a preferred enantiomer of a 2-hydroxycarboxylic acid may be delivered to topical surfaces. For example, the topical formulations may be for topical application to the skin, mucosal membranes (oral, nasal, vaginal, rectal) or eye of the subject.

The compositions of the present invention may contain water (aqueous) or may be non-aqueous.

The composition of this invention may also include minor amounts of conventional additives such as viscosity modifiers, for example xanthan gum, and preservatives, such as phenoxyethanol or benzyl alcohol, including mixtures thereof. For some therapeutic agents it may be necessary to incorporate buffering agents to maintain a suitable pH.

Suitable preservatives for use in such a composition or medicament include, for example, phenoxyethanol, and other preservatives conventionally used in pharmaceutical preparations, especially in creams. Suitable preservatives include methyl hydroxybenzoate, chlorocresol, sorbic acid and benzoic acid.

The compositions of the invention may be produced by conventional pharmaceutical techniques. Thus, ointments and creams are conveniently prepared by mixing together at an elevated temperature, preferably 60-70° C., the components constituting the vehicle until an emulsion has formed. The mixture may then be cooled to room temperature, and, after addition of the preferred enantiomer of a 2-hydroxycarboxylic acid, together with any other ingredients, stirred to ensure adequate dispersion.

Liquid preparations, such as ear and eye drops, are produced by dissolving the therapeutic agent in the components constituting the vehicle and the other ingredients are then added. The resulting solution or suspension is distributed into glass or plastic bottles or in single dose packs such as soft gelatine capsules which are then heat sealed.

Artificial tear vehicles may be used for ocular compositions comprising a preferred enantiomer of a 2-hydroxycarboxylic acid delivery. More viscous artificial tears use high concentrations of viscosity enhancing agents, such as Celluvisc®, high viscosity carboxymethyl cellulose (CMC) and Refresh Liquigel®, a blend of 0.35% high viscosity CMC and 0.65% low viscosity CMC.

Gelling agents may be used for compositions comprising a preferred enantiomer of a 2-hydroxycarboxylic acid being delivered to the eye. Such agents may be instilled as liquid and then almost immediately triggered to a gel phase. Timoptic gel (gellan gum), AzaSite® (polycarbophil, poloxamer), and Besivance®, (polycarbophil, poloxamer), 0.3% alginate Keltrol® are examples of such agents. Another gelling agent is polycarbophil-poloxamer gels (eg Durasite®).

The ocular carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminium monostearate and gelatine.

Nasal delivery of a preferred enantiomer of a 2-hydroxycarboxylic acid is also contemplated. Nasal delivery allows the passage of the preferred enantiomer of a 2-hydroxycarboxylic acid to the blood stream directly after administering the therapeutic product to the nose, without the necessity for deposition of the preferred enantiomer of a 2-hydroxycarboxylic acid in the lung. Compositions for nasal delivery include those with dextran or cyclodextran.

### Oral Delivery Excipients

The compositions of the present invention comprising a preferred enantiomer of a 2-hydroxycarboxylic acid may be delivered orally. For example, the oral formulations may be for local application to specific parts of the gastrointestinal system (such as the oesophagus, stomach or large or small intestines) or for a systemic application and may be delivered in a multitude of forms including a simple solution, syrup, suspension, tablet or capsule.

The compositions of the present invention may contain water (aqueous) or may be non-aqueous.

The composition of this invention may also include minor amounts of conventional additives such as preservatives, solubilizers (including ethanol), complexing agents (such as cyclodextrin), flowability enhancers and glidants (including colloidal silica), flavour enhancers and compactibility enhancers, including mixtures thereof. For some therapeutic agents it may be necessary to incorporate buffering agents to maintain a suitable pH.

The oral compositions of the present invention may be delivered with an immune boosting or modulating agents. Examples of immune boosting or modulating agents include specific immunostimulants (such as vaccines and antigens) and non-specific agents such as honeybee products (including propolis and honey), pro- and pre-biotics, hormones, vitamins (vitamin c, vitamin d), minerals (zinc oxide), antioxidants (including glutathione), interferons (including INF-alpha), interleukins (including interleukin 10), colchicine, thalidomide and imiquimod. The immune booster may be delivered at the same time as the preferred enantiomer of a 2-hydroxycarboxylic acid composition, or after the preferred enantiomer of a 2-hydroxycarboxylic acid composition.

Without being held to any theory, it is believed that administration of the preferred enantiomer of a 2-hydroxycarboxylic acid may sufficiently disrupt biofilms in the body such that concurrent or subsequent administration of the immune boosting agent allows the body to clear the bacteria in the disrupted biofilm without the use of further agents such as antibiotics.

### Stability

Preferably, the compositions of the present invention are stable. As used herein, the stability of compositions provided herein refers to the length of time at a given temperature that greater than 80%, 85%, 90% or 95% of the initial amount of antibiotic-lactic acid, is present in the composition. For example, the compositions provided herein may be stored between about 15° C. and about 30° C., and remain stable for at least 1, 2, 12, 18, 24 or 36 months. Also, the compositions may be suitable for administration to a subject in need thereof after storage for more than 1, 2, 12, 18, 24 or 36 months at 25° C. Also, in another alternative embodiment, using Arrhenius Kinetics, more than 80%, or more than 85%, or more than 90%, or more than 95% of the initial amount of active (e.g., the preferred enantiomer of a 2-hydroxycarboxylic acid) remains after storage of the compositions for more than 1, 2, 12, 18, 24 or 36 months between about 15° C. and about 30° C.

As used herein, the statement that a composition is stable during “long term storage” means that the composition is suitable for administration to a subject in need thereof when it has an estimated shelf-life of greater than 1, 2 or 3 months usage time at 25° C. and greater than or equal to 1, 2 or 3 years storage time at 5° C. In certain embodiments herein, using Arrhenius kinetics, an estimated >80% or >85% or >90% or >95% of the preferred enantiomer of a 2-hydroxycarboxylic acid remains after such storage.

### Antimicrobials

Other active agents may also be incorporated into the composition of the present invention. For example, additional antimicrobial agents such as antibacterials, antifungals etc may be incorporated.

The additional active agent provided with the preferred enantiomer of a 2-hydroxycarboxylic acid of the present invention may be an active agent that kills bacteria, but is not an antibiotic. For example, the additional active agent may be a chlorine-based compound (such as bleach), an iodine-based compound, a copper-based compound, etc. The antimicrobial compound may be an agent with a different mode of antimicrobial action, such as a bacteriophage, antiseptic, saline, chlorine-based compound (such as bleach), an iodine-based compound, a copper-based compound, or heavy metal.

The composition may further comprise benzoyl peroxide, or an antibiotic such as erythromycin, clindamycin, doxycycline or meclocycline.

Additional antimicrobial agents that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin, azaserine, candicidin(s), chlorphenesin, dermostatin(s), filipin, fungichromin, mepartricin, nystatin, oligomycin(s), ciproflaxacin, norfloxacin, ofloxacin, pefloxacin, enoxacin, rosoxacin, amifloxacin, fleroxacin, temafloaxcin, lomefloxacin, perimycin A or tubercidin, and the like.

### Use

The present invention provides for the use of a preferred enantiomer of a 2-hydroxycarboxylic acid a preferred 2-hydroxycarboxylic acid in the manufacture of a composition for the disruption of a biofilm.

The present invention further provides for the use of a preferred enantiomer of a 2-hydroxycarboxylic acid for the disruption of a biofilm.

The present invention provides for the use of a preferred enantiomer of a 2-hydroxycarboxylic acid in the manufacture of a composition for the disruption of a biofilm in combination with an antimicrobial compound.

The present invention further provides for the use of a preferred enantiomer of a 2-hydroxycarboxylic acid for the disruption of a biofilm in combination with an antimicrobial compound.

The present invention further provides for the use of a preferred 2-hydroxycarboxylic acid in the manufacture of a composition for treating or preventing an infection in a subject wherein the infection is associated with a biofilm and wherein the composition disrupts the biofilm.

The present invention further provides for the use of a preferred enantiomer of a 2-hydroxycarboxylic acid in the manufacture of a composition for treating or preventing an infection in a subject, in combination with an antimicrobial compound, wherein the infection is associated with a biofilm and wherein the composition disrupts the biofilm.

The present invention further provides for the use of a preferred enantiomer of a 2-hydroxycarboxylic acid for treating or preventing an infection in a subject wherein the infection is associated with a biofilm and wherein the composition disrupts the biofilm.

The present invention further provides for the use of a preferred enantiomer of a 2-hydroxycarboxylic acid for treating or preventing an infection in a subject, in combination with an antimicrobial compound wherein the infection is associated with a biofilm and wherein the composition disrupts the biofilm.

The present invention further provides for the use of a preferred enantiomer of a 2-hydroxycarboxylic acid for disrupting or preventing formation of a biofilm on a non-living surface.

The present invention further provides for the use of a preferred enantiomer of a 2-hydroxycarboxylic acid for disrupting or preventing formation of a biofilm on a non-living surface, in combination with an antimicrobial compound.

The present invention further provides for the use of a preferred enantiomer of a 2-hydroxycarboxylic acid in the manufacture of a composition for treating or preventing microbial colonisation of a surface wherein the microbial colonisation is associated with a biofilm and wherein the composition disrupts the biofilm.

The present invention further provides for the use of a preferred enantiomer of a 2-hydroxycarboxylic acid in the manufacture of a composition for treating or preventing microbial colonisation of a surface, in combination with an antimicrobial compound, wherein the microbial colonisation is associated with a biofilm and wherein the composition disrupts the biofilm.

The present invention further provides for the use of a preferred enantiomer of a 2-hydroxycarboxylic acid for treating or preventing microbial colonisation of a surface wherein the microbial infection is associated with a biofilm and wherein the composition disrupts the biofilm.

The present invention further provides for the use of a preferred enantiomer of a 2-hydroxycarboxylic acid for treating or preventing microbial colonisation of a surface, in combination with an antimicrobial compound wherein the microbial colonisation is associated with a biofilm and wherein the composition disrupts the biofilm.

The preferred enantiomer of a 2-hydroxycarboxylic acid may be in the same composition as the antimicrobial compound, or may be in separate compositions. The antimicrobial compound may be an antibiotic, or agent with a different mode of antimicrobial action, such as a bacteriophage, antiseptic, saline, chlorine-based compound (such as bleach), an iodine-based compound, a copper-based compound, or heavy metal. The biofilm may be an infection of a subject, or may be colonisation of a surface, such as a non-living surface. The biofilm may be a free-floating biofilm-associated aggregate of bacteria.

If the preferred enantiomer of a 2-hydroxycarboxylic acid is in a separate composition to the antimicrobial compound, the preferred enantiomer of a 2-hydroxycarboxylic acid may be administered at the same time as the antimicrobial compound, or may be administered at a different time as the antimicrobial compound.

For the above uses, the preferred enantiomer of a 2-hydroxycarboxylic acid may be D-lactic acid. Preferably the infection or colonisation is infection or colonisation by a bacteria.

### Kits

The present invention provides a kit for the disruption of a biofilm comprising:
a) a preferred enantiomer of a 2-hydroxycarboxylic acid; and
b) instructions for use.

The present invention provides a kit for the disruption of a biofilm comprising:
a) a preferred enantiomer of a 2-hydroxycarboxylic acid; and
b) instructions for use
   wherein the preferred enantiomer of a 2-hydroxycarboxylic acid is in combination with an antimicrobial compound.

The preferred enantiomer of a 2-hydroxycarboxylic acid of the kit may be D-lactic acid.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the composition may be applied to an affected area of the animal, such as the lungs, injected into an animal, or even applied to and mixed with the other components of the kit.

In an embodiment, the kit of the present invention comprises a composition comprising a preferred enantiomer of a 2-hydroxycarboxylic acid, optionally in combination with an antimicrobial agent. In an alternative embodiment, the composition is in pre-measured, pre-mixed and/or pre-packaged.

The kit of the present invention may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present invention comprises instructions for administering the compositions of the present invention. In one embodiment, the instructions indicate that the composition of the present invention is suitable for the disruption of biofilms. Such instructions may also include instructions on dosage, as well as instructions for administration.

The preferred enantiomer of a 2-hydroxycarboxylic acid and suitable excipients can be packaged individually so to allow a practitioner or user to formulate the components into a pharmaceutically acceptable composition as needed. Alternatively, the antisense oligomers and suitable excipients can be packaged together, thereby requiring de minimis composition by the practitioner or user. In any event, the packaging should maintain chemical, physical, and aesthetic integrity of the active ingredients.

### General

Those skilled in the art will appreciate that the invention described herein is susceptible to variations and modifications other than those specifically described. The invention includes all such variation and modifications. The invention also includes all of the steps, features, compositions and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, compositions and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Application of Prebiotic Derived Agents for Biofilm Disruption

This study demonstrates that a prebiotic solution derived from cultures that were propagated from a commercially available probiotic capsule disrupts biofilm-protected bacterial microcolonies. The extent of the disruption was found to be comparable to that caused by the gold standard antibiotic therapy prescribed to treat this bacterial strain.

### Materials and Methods

### Prebiotic Test Solution Preparation

The powdered contents of a commercially available multi-strain probiotic capsule (45 billion CFU, see Table 1 for contents) was added to 10 ml of Lysogeny broth (LB) and cultured (initial culture, C₀) overnight at 37° C., 5% CO₂.

TABLE 1
Bacteria present in multi-strain probiotic capsule

| Bacterial strain | Bacterial titre per capsule |
|---|---|
| Lactobacillus rhamnosus | 15.55 billion CFU |
| Lactobacillus casei | 9.45 billion CFU |
| Lactobacillus acidophilus | 7.4 billion CFU |
| Lactobacillus plantarum | 3.15 billion CFU |
| Lactobacillus fermentum | 1.35 billion CFU |
| Bifidobacterium lactis | 4.05 billion CFU |
| Bifidobacterium breve | 1.35 billion CFU |
| Bifidobacterium bifidum | 450 million CFU |
| Streptococcus thermophilus | 2.25 billion CFU |
| Total per capsule | 45 billion CFU |

The culture was propagated to stationary phase. Stationary phase refers to the time in a closed culture system when the number of new cells being produced in a culture is equal to the number of cells dying off. This occurs directly after the exponential growth phase due to nutrient depletion and build-up of waste products. During stationary phase bacteria remain metabolically active but overall culture growth has ceased resulting in a flattening out of the growth curve.

Cultures were propagated to stationary phase to induce production of secondary metabolites (metabolites produced after active growth, such as antibiotics); substrates generated from this phase were predicted to have possible biofilm disruption ability. Stationary phase culture (C₁) was achieved by looping from the initial culture (C₀) into 5 ml of LB broth and incubating overnight at 37° C., 5% CO₂ producing a resulting culture C₁. The resulting multi-strain bacterial culture and prebiotic test solution collected from C₁ were both assessed for their ability to breakdown the biofilm matrix of biofilm-protected bacterial microcolonies.

### Biofilm-Protected Bacterial Microcolony Culture

Laboratory strains of Pseudomonas aeruginosa (WACC91 and PA-001) were thawed (stored as 50% glycerol stocks at −70° C.+2° C.) and streaked out on blood agar plates. These were allowed to grow at 37° C., 5% CO₂ for 12-24 hours until isolated colonies were large enough to sustain further culturing could be selected. Isolated colonies were looped into 5 ml of LB broth and incubated overnight at 37° C., 5% CO₂ with gentle shaking to achieve stationary phase (B₀). In this case cultures were propagated to stationary phase to induce stress responses needed to induce biofilm formation on seeding for treatment.

B₀ cultures were diluted 1:1000 in Pseudomonas minimal medium with 2% arginine (herein referred to as PMM) to achieve approximately 10⁸ cfu/ml (confirmed by measuring 0.02 absorbance units at 600 nm, approximately 1.8 ul in 2 ml of PMM). Diluted B₀ cultures were mixed well and seeded at 200 ul/well across a 96 well microtitre plate. Biofilm formation was complete approximately 24-48 hours following seeding, this was confirmed by a minimum of 1.8 absorbance units at 600 nm.

### Biofilm Disruption Treatment Preparations

Prebiotic solution collected from C₁ was applied to B₀ cultures that had achieved biofilm formation. A 10 dose dilution series of the prebiotic test solution in PMM was prepared (Table 2) to determine the optimal dose range for biofilm disruption of B₀ cultures, with 50 μl total volume added to each B₀ culture.

TABLE 2
10 dose dilution series of the prebiotic test solution in PMM
Prebiotic test solution dilution series

| | stock | final dilution | stock | buffer | final | treatment |
|---|---|---|---|---|---|---|
| | dilution | factor of prebiotic | volume | volume | volume | volume |
| stock | factor | test (treatment) | (ul) | (ul) | (ul) | (ul) |
| 1 in 4 | 2 | 8 | 100 | 100 | 200 | 50 |
| dilution of | 3 | 12 | 67 | 133 | 200 | 50 |
| prebiotic test | 4 | 16 | 50 | 150 | 200 | 50 |
| | 5 | 20 | 40 | 160 | 200 | 50 |
| | 6 | 24 | 33 | 167 | 200 | 50 |
| | 7 | 28 | 29 | 171 | 200 | 50 |
| | 8 | 32 | 25 | 175 | 200 | 50 |
| | 9 | 36 | 22 | 178 | 200 | 50 |
| | 10 | 40 | 20 | 180 | 200 | 50 |
| | 11 | 44 | 18 | 182 | 200 | 50 |

Tobramycin dilutions

| | | | rounded |
|---|---|---|---|
| | | | final conc |
| Dilution | Dilution | final conc | on graph |
| number | factor | (ug/ml) | (ug/ml) |
| 1 | 2 | 16 | 16 |
| 2 | 2 | 8 | 8 |
| 3 | 2 | 4 | 4 |
| 4 | 2 | 2 | 2 |
| 5 | 2 | 1 | 1 |
| 6 | 2 | 0.5 | 0.5 |
| 7 | 2 | 0.25 | 0.25 |
| 8 | 2 | 0.125 | 0.13 |
| 9 | 2 | 0.0625 | 0.06 |
| 10 | 2 | 0.03125 | 0.03 |
| 11 | 2 | 0.015625 | 0.02 |
| 12 | 2 | 0.0078125 | 0.01 |

A 12-fold serial dilution of tobramycin (99% pure tobramycin sulfate powder, Research Products International) standards were prepared in PMM (8 ng/ml-16 ug/ml), to act as a reference point for assessing biofilm disruption activity observed by the prebiotic test treatment (Table 2). Tobramycin is an antibiotic commonly used to treat chronic respiratory bacterial infections, particularly Pseudomonas aeruginosa. A total of 50 ul of tobramycin standards were added to B₀ cultures that had achieved biofilm formation. The target concentration of bioactive tobramycin within the epithelial lining fluid of paediatric patients with cystic fibrosis is 20 μg/ml (Rosenthal et al 2001), however as antibiotic resistance breaking point for tobramycin is 16 ug/ml, this is the maximum dose used across studies investigating tobramycin activity (Clinical and Laboratory Standards Institute, 2005).

### Biofilm Disruption Evaluation

i. Crystal Violet Staining

Biofilms were stained with 0.1% w/v of crystal violet (CV) in water to each well of the microtiter plate after plate wells were cleared and washed by submersion in water (O'Toole 2011). Following incubation at room temperature for 15 minutes the microtiter plates were washed 3-4 times with water by submerging in water, with blotting to rid the plate of all excess cells and dye. Plates were left to dry overnight. The CV stain was solubilised with 125 ul of 30% acetic acid and absorbance to indicate biofilm density (also referred to as biofilm biomass) was quantified at 550 nm following an incubation of 15 minutes at room temperature.

ii. Resazurin Staining

Resazurin stain was used to measure changes in bacterial metabolic activity following treatment (Kirchner et al 2012). 10 μl of 0.02% (v/v) resazurin (diluted in distilled water) was added to each well and the microtite plates incubated under aerobic conditions for 1-2 h at 37° C., while shaking at 150 rpm. Viable cells reduce the blue resazurin dye to the pink, fluorescent resorufin form. Following incubation fluorescence was quantified by excitation wavelength of 540 nm and an emission wavelength of 590 nm using Enspire microplate reader.

### Statistical Analysis

All data presented represents a minimum of 12 independent experimental replicates containing technical duplicates unless otherwise stated. Students t-test used to assess significance compared to control untreated biofilms with significance measured at p<0.05.

### Results

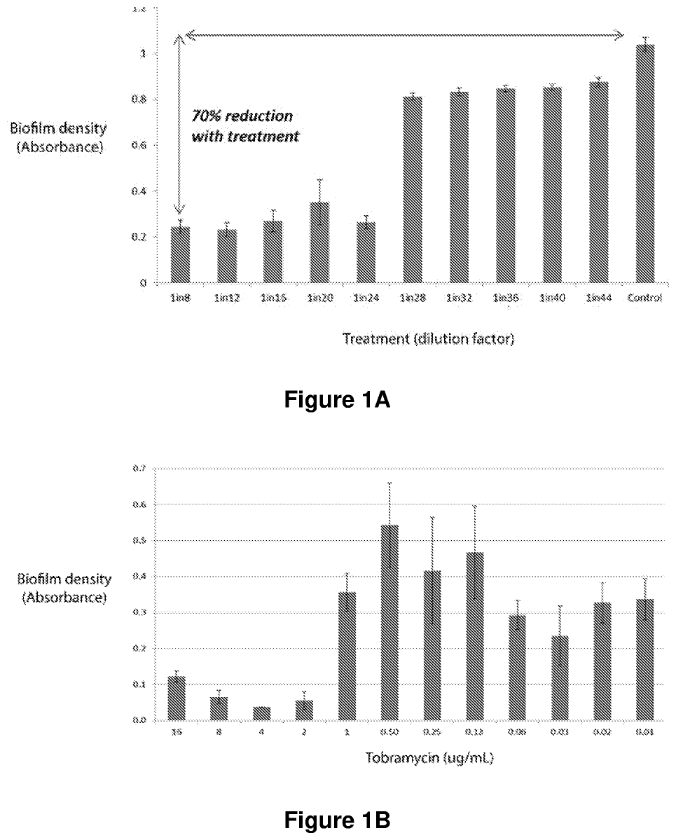

The five most concentrated dilutions of the prebiotic test solution induced 70-80% reduction in crystal violet staining of WACC91 and PA01 laboratory strains of P. aeruginosa biofilms compared to non-treated biofilms (n=12 biological replicates, FIG. 1). This result indicated a significantly reduced biofilm density compared to untreated biofilm cultures within the same experiment (n=12, p<0.0001).

The reductions in biofilm density induced by the prebiotic test solution were comparable to reduced density (80-90%) observed in the same biofilm-protected P. aeruginosa microcolonies treated with the antibiotic tobramycin (16 ug/ml, bioactive dose). This demonstrates that the prebiotic test solution was capable of reducing biofilm density at an efficacy approaching that achieved by the bioactive dose accepted for tobramycin; the antibiotic most commonly prescribed to treat aggressive P. aeruginosa infections.

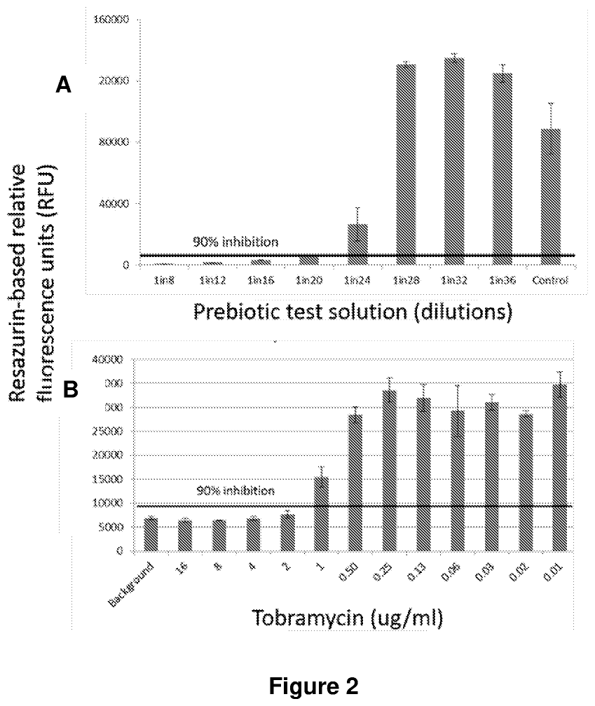

Resorufin fluorescence was measured to quantify the impact of the prebiotic solution and tobramycin on the metabolic activity of biofilm-protected cultures. 90% inhibition of metabolic activity was observed for cultures treated with prebiotic test solution for the four most concentrated dilutions (FIG. 2). This was comparable to inhibition observed for bioactive dose of tobramycin (16 ug/ml) indicating a significant interruption of bacterial activity when treated with the prebiotic solution.

Prebiotic test solution is able to reduce P. aeruginosa biofilm biomass as effectively as the most commonly prescribed antibiotic for aggressive P. aeruginosa infections and has a significant impact on baseline metabolic activity.

### Example 2

### D-Lactic Acid is an Active Biofilm Disruptor

The active agent causing the biofilm disruption in Example 1 was investigated by testing biofilm disruption of biofilm-protected P. aeruginosa (WACC91) microcolonies in response to HPLC fractionations until a single peak was identified. Mass spectrometry and nuclear magnetic resonance were used to identify the contents of the single peak.

### Materials and Methods

### HPLC Fractionation

Bioactivity analysis of incremental ethanol solute fractions determined by high performance liquid chromatography (HPLC) was performed until resolution of a single compound was reached.

### Bioassay to Assess Biofilm Disruption of Prebiotic HPLC Fractions

### Biofilm Microcolony Culture

Laboratory strains of Pseudomonas aeruginosa WACC91 were thawed and streaked out on blood agar plates and allowed to grow at 37° C., 5% CO₂ for 12-24 hours until suitable growth was achieved to enable further culturing. Isolated colonies (W₀) were looped into 5 ml of LB broth and incubated overnight at 37° C., 5% CO₂ with gentle shaking to achieve stationary phase.

W₀ cultures were diluted 1:1000 in Pseudomonas minimal medium with 2% arginine (herein referred to as PMM) to achieve approximately 10⁸ cfu/ml (confirmed by measuring 0.02 absorbance units at 600 nm, approximately 1.8 ul in 2 ml of PMM). Diluted W₀ cultures were mixed well and seeded at 200 ul/well across a 96 well microtitre plate. Biofilm formation was complete approximately 24-48 hours following seeding as confirmed by a minimum of 1.8 absorbance units at 600 nm.

### Treatments

HPLC fractions were added to biofilm microcolonies to assess biofilm disruption activity after overnight treatment at 37° C., 5% CO₂ (50 ul added to each culture). Each HPLC fraction treatment was run in duplicate.

### Evaluation of Biofilm Disruption

i. Crystal Violet Staining

Biofilms were stained with 0.1% w/v of crystal violet (CV) in water to each well of the microtiter plate after the plate wells were cleared and washed by submersion in water (O'Toole 2011). Following incubation at room temperature for 15 minutes the microtiter plates were washed 3-4 times with water by submerging in water, with blotting to rid the plate of all excess cells and dye. Plates were left to dry overnight. The CV stain was solubilised with 125 ul of 30% acetic acid and absorbance to indicate biofilm biomass quantified at 550 nm following an incubation of 15 minutes.

### Bioactivity Analysis

A minimum of two bioactivity assays were completed for each HPLC fractionation. With untreated control samples, assay controls and technical duplicates included for each evaluation test.

### Nuclear Magnetic Resonance

Nuclear magnetic resonance (NMR) was used to identify the compound in the final HPLC peak.

### Results

Prebiotic test solution was dissolved in ethanol with aqueous and non-aqueous extracts assessed for biofilm bioactivity. Biofilm disruption was observed for the aqueous extract only, eliminating identification of a small protein biofilm disruptor.

Fractionation of the ethanol aqueous extract by HPLC revealed biofilm disruption activity in a single fraction (FIG. 2). Mass spectrometry and nuclear magnetic resonance (NMR) of this fraction identified lactic acid as the single constituent of this main peak.

Optical rotation analysis of this compound showed 95% of the contents to be D-lactic acid; the rarer isomer form of lactic acid (FIG. 2).

Pure grade D-Lactic acid standards were prepared (Sigma ThermoFisher) and run against prebiotic test solution dilutions used to treat biofilm-protected microcolonies (using the methods of Example 1) to determine the active dose range for D-Lactic acid. This was determined at a range of 0.25-1.25 mg/ml.

D-lactic acid is the active agent in probiotic extract that reduces P. aeruginosa biofilm density at an active dose range of 0.25-1.25 mg/ml.

### Example 3

### D-Lactic Acid Biofilm Disruption Activity is not Toxic to Human Cells

The bactericidal activity, cytotoxicity of D-Lactic acid to human cells and impact of pH on D-Lactic acid biofilm disruption activity were assessed.

The resorufin fluorescence data from Example 1 indicated an impact of D-Lactic acid on bacterial metabolic activity, however it was unclear from this data if this was bactericidal. A bacterial bioassay designed for high-throughput screening of antibiotics was used to assess bactericidal activity of D- and L-lactic acid as outlined below. The ability of D-Lactic acid to remain active at physiological pH without causing cytotoxicity are key data points to demonstrate therapeutic potential of D-Lactic acid.

### Materials and Methods

### Bactericidal Activity

Antimicrobial activity of D- and L-lactic acid was assessed by quantifying changes in planktonic (non-biofilm) bacterial cell counts and phenotype of E. coli (ATCC 25922), P. aeruginosa (ATCC 27853), and S. aureus (ATCC 33592) upon treatment to a concentration gradient using flow cytometry (FCM) and a modified FAST analytical pipeline. This was undertaken in parallel with a conventional broth microdilution (BMD) assay. The minimum inhibitory concentration (MIC) for D- and L-lactic acid was found to be 4 g/L for all strains.

Two-fold serial dilutions of D- or L-lactate were performed in a 96-well plate, ranging from 0.008 grams per litre to 8 g/L with a volume of 50 μL per well. Each replicate dilution series had an additional control well containing 50 μL assay media (CAMHB) without lactic acid, serving as an unexposed control.

The bacterial suspensions were prepared from cultures grown overnight in trypticase soya broth (TSB). Each culture was diluted 1:1000 in Hanks buffered saline solution (HBSS), stained with 1 μL of SYTO® 9, incubated for a minimum of 8 minutes with shaking, and enumerated by flow cytometry. The TSB cultures were then adjusted to 2×10⁵ cells per mL in CAMHB.

To the replicate dilution series of lactic acids, 50 μL of bacterial suspension was added for a final volume of 100 μL per well, each row containing a concentration gradient ranging from 0.004 to 4 g/L. There were 3 organisms per plate (E. coli, P. aeruginosa, S. aureus), set up in duplicate. The plates also contained three wells containing 50 μL of CAMHB and 10 μL 12% formalin, to which 50 μL of each bacterial suspension were individually added. These wells were representative of the suspension density at the time of inoculation.

The plates were incubated at 35.5° C. For conventional BMD, the incubation time was 24 hours. For flow cytometry, the incubation time was 3 hours. For flow cytometry, following incubation 100 μL of HBSS, containing SYTO® 9 at a concentration of 10 μM was added to each well of the plate for a final dye concentration of 5 μM. The plates were then incubated at ambient temperature in the dark for 10 minutes, loaded onto the Attune™ Autosampler, and the data acquired. The plates were run simultaneously on two different Attune™ cytometers.

### Cytotoxicity of D-Lactic Acid

D-lactic acid cytotoxicity was assessed on transformed human airway epithelial cell cultures. A549 and BEAS-2B human airway epithelial cells were seeded at 60,000 cells/ml and grown to 70% confluence in Dulbecco's modified eagle's medium (DMEM) and supplemented with 10% fetal calf serum with 0.05% gentamicin.

The active dose range observed for biofilm disruption (0.5-1.25 mg/ml) was used to assess cytotoxic effects by microscopy of cellular exclusion of trypan blue after 24 hours. Briefly, 50 ul of D-lactic acid doses of 0.5, 1 and 1.25 mg/ml were added to cell cultures which were than incubated for 24 hours at 37° C. in 5% CO₂. Following incubation, 50 μl of 0.4% (w/v in ethanol) trypan blue solution was added to each well and incubated for 10 minutes at room temperature. As trypan blue permeates the compromised cell walls of dead cells, trypan blue positive cells are identified as dead cells that can be counted and used to quantify overall portion of dead cells in the culture for treatment comparisons.

### Impact of pH on D-Lactic Acid Ability to Disrupt Biofilms

### Biofilm Microcolony Culture

Laboratory strains of Pseudomonas aeruginosa WACC91 were thawed and streaked out on blood agar plates and allowed to grow at 37° C., 5% CO₂ for 12-24 hours until suitable growth was achieved to enable further culturing. Isolated colonies (W₀) were looped into 5 ml of LB broth and incubated overnight at 37° C., 5% CO₂ with gentle shaking to achieve stationary phase.

W₀ cultures were diluted 1:1000 in Pseudomonas minimal medium with 2% arginine (herein referred to as PMM) to achieve approximately 10⁸ cfu/ml (confirmed by measuring 0.02 absorbance units at 600 nm, approximately 1.8 ul in 2 ml of PMM). Diluted W₀ cultures were mixed well and seeded at 200 ul/well across a 96 well microtitre plate. Biofilm formation was complete approximately 24-48 hours following seeding as confirmed by a minimum of 1.8 absorbance units at 600 nm.

Treatment with D-Lactic Acid Buffered to pH 2.4, 7

D-Lactic acid stock solution was buffered to pH 2.4 with 5M hydrochloric acid and to pH 7 with 5M sodium hydroxide prior to dilution in PMM for use.

### Evaluation of Biofilm Disruption

i. Crystal Violet Staining

Biofilms were stained with 0.1% w/v of crystal violet in water to each well of the microtiter plate after the plate wells were cleared and washed by submersion in water (O'Toole 2011). Following incubation at room temperature for 15 minutes the microtiter plates were washed 3-4 times with water by submerging in water, with blotting to rid the plate of all excess cells and dye. Plates were left to dry overnight. The CV stain was solubilised with 125 ul of 30% acetic acid and absorbance to indicate biofilm biomass quantified at 550 nm following an incubation of 15 minutes.

### Statistical Analysis

All data presented represents a minimum of 3 independent experimental replicates containing technical duplicates unless otherwise stated. Students t-test used to assess significance compared to control untreated biofilms with significance measured at p<0.05.

### Results

### Bactericidal Activity

L- and D-lactic acid induced similar trends in phenotypic changes and overall event counts for E. coli, P. aeruginosa, S. aureus. After correction for time dependent growth (some growth can occur during data acquisition), we observed a proportional increase in bacterial event counts with increasing concentrations for both compounds up to 0.5 g/L. These data suggest that both compounds have a stimulatory effect on bacterial growth from 0.008 g to 0.5 g per litre.

No significant phenotypic change was observed in E. coli, P. aeruginosa, S. aureus until 1 g/L, at which a significant decrease in SYTO® 9 fluorescence was observed, with no change in forward scatter. This trend continued until 2 g/L. At 2 g/L we observed a general decrease in total bacterial counts. This trend continued at 4 g/L where we observed another sharp decrease in bacterial counts coinciding with a very large magnitude shift in the bacterial population, decreasing in both mean forward scatter (FSC) and SYTO®9 fluorescence. The distribution of the population by SYTO® 9 fluorescence increases, with the cluster losing cohesion and becoming more diffuse. This change in phenotype is consistent with the occurrence of early or incomplete cellular lysis, suggesting toxic effects of D- and L-isomers at doses above 4 g/L for E. coli, P. aeruginosa, S. aureus.

### Toxicity

No significant differences between treated and untreated control cell cultures (data not shown).

Acute toxicity data for D-Lactic acid taken from Fisher Scientific MSDS suggests the active dose range of 0.25-1.25 mg/ml is below the LD50 doses observed for oral, dermal and inhaled toxicology doses observed in vivo (as measured in rat and rabbits (Table 3).

TABLE 3
Toxicity of lactic acid (Fisher Scientific MSDS for D-Lactic acid)

| Component | LD50 Oral | LD50 Dermal | LC50 Inhalation |
|---|---|---|---|
| Lactic acid | 3543 kg (rat) | >2 g/kg (rabbit) | >7.94 mg/L (rat) 4 h |

### Impact of pH on D-Lactic Acid Ability to Disrupt Biofilms

Biofilm disruption activity of D-Lactic acid observed at pH 2.4 was maintained at pH 7 (physiological pH), indicating activity of D-Lactic acid is independent of pH (data not shown). D-Lactic acid buffered to pH 6 used as part of standard treatment protocol from this point forward.

In summary, D-lactic acid is not bactericidal in the active biofilm disruption dose range (0.5-1.25 mg/ml). The active biofilm disruption dose range active dose range for D-lactic acid is not cytotoxic. Biofilm disruption activity of D-Lactic acid is not affected by pH.

### Example 4

D-Lactic Acid Breaks Down Bacterial Biofilms Derived from a Range of Bacterial Strains Including Antibiotic Resistant and Non-Resistant Clinical Isolates

The efficacy of D-lactic acid to disrupt biofilms was tested across 24 clinical isolates from patients with cystic fibrosis with known antibiotic resistance profiles. Laboratory strains of Non-typable Haemophilus influenzae (NTHi) and Actinobacillus pleuropneumoniae (App), these are the most prevalent pathogens for human otitis media and porcine respiratory syndrome respectively and were included for analysis.

### Materials and Methods

### Clinical Isolates

These were sourced by the investigators from a biobank of cultured extracts grown from bronchoalveolar lavage collected from patients with cystic fibrosis held at PathWest Laboratory Services. These extracts are collected by physicians for diagnostic purposes, whereby the presence of antibiotic resistant pathogens is identified by PathWest to inform clinical diagnoses and patient treatment pathways.

### Biofilm Microcolony Culture

Cultures of interest were thawed and streaked out on blood agar plates and allowed to grow at 37° C., 5% CO₂ for 12-24 hours until suitable growth was achieved to enable further culturing. An isolated colony was looped into 5 ml of LB broth and incubated overnight at 37° C., 5% CO₂.

Stock cultures were diluted 1:1000 in 1% of appropriate media (LB for mucoid P. aeruginosa strains; M63 medium was used for P. aeruginosa rough strains 001-11R; PMM for all other strains) to achieve approximately 10⁸ cfu/ml (confirmed by measuring 0.02 absorbance units, approximately 1.8 ul in 2 ml of media). Diluted working cultures were mixed well and seeded at 200 ul/well across a 96 well microtitre plate, with biofilm formation complete approximately 24-48 hours following seeding.

### Treatments

D-lactic acid was applied to all bacterial biofilm microcolonies at 0.5-1.25 ug/ml for 24 hours. Treatments were completed in duplicate for all bacterial strains across the dose range used. Tris and hydrochloric acid were used to prepare buffered solutions of at pH 2.4 to pH 7 of D-lactic acid at 0.5 mg/ml to assess any pH dependencies of biofilm disruption activity.

### Biofilm Disruption Evaluation

i. Crystal Violet Staining

Biofilms were stained with 0.1% w/v of crystal violet (CV) in water to each well of the microtiter plate after the plate wells were cleared and washed by submersion in water (O'Toole 2011). Following incubation at room temperature for 15 minutes the microtiter plates were washed 3-4 times with water by submerging in water, with blotting to rid the plate of all excess cells and dye. Plates were left to dry overnight. The CV stain was solubilised with 125 ul of 30% acetic acid and absorbance to indicate biofilm biomass quantified at 550 nm following an incubation of 15 minutes.

ii. Resazurin Staining

Resazurin stain was used to measure changes in bacterial metabolic activity following treatment (Kirchner et al 2012). 10 μl of 0.02% (v/v) resazurin (diluted in distilled water) was added to each well and the microtiter plates incubated under aerobic conditions for 1-2 h at 37° C., while shaking at 150 rpm. Viable cells reduce the blue resazurin dye to the pink-fluorescent resorufin form. Following incubation fluorescence was quantified by excitation wavelength of 540 nm and an emission wavelength of 590 nm using Enspire microplate reader.

### Results

Greater than 50% of all strains showed evidence of biofilm disruption following treatment with D-lactic acid. More than 65% of all strains tested showed a change in metabolic activity following D-lactic acid treatment (Table 4).

TABLE 4
Response summary of biofilm-protected microcolonies
established from clinical isolate responses to D-Lactic acid.

| | Mucoid | Non-mucoid |
|---|---|---|
| Number of P. aeruginosa | 11 | 13 |
| clinical isolates | | |
| Isolates responsive to | 60 | 53.8 |
| breakdown (% of isolates) | | |
| Biofilm breakdown (% from baseline) | 17.2 | 23 |
| Altered metabolism with | 75 | 69.2 |
| treatment (% of isolates) | | |
| Metabolism change (% from baseline) | 37 | 46.1 |

Bold = % where there was a significant difference to controls.
Not bold = % that displayed a response that could not be quantified relative to controls eg. variations not tolerated by the assay for quantification.

This suggests a change in biofilm morphology may have been observed with ongoing D-lactic acid treatment outside of the controlled conditions implemented for experimental rigor.

Biofilm disruption was evident in biofilm-protected NTHi and App microcolonies (data not shown). This is significant as NTHi is commonly associated with otitis media (ear infections) and App is a problematic pathogen in commercial pig farms indicating broad spectrum biofilm disruptive activity of D-Lactic acid.

D-lactic acid will reduce P. aeruginosa biofilms that are antibiotic resistant, mucoid or non-mucoid. D-lactic acid will reduce biofilms grown from other bacteria known to induce clinical illness. D-lactic acid can be considered a broad-spectrum bacterial biofilm disruptor.

### Example 5

### D-Lactic Acid Breaks Down Biofilms and Induces Antimicrobial Activity Similar to Antibiotic Therapy

Crystal violet is the widely recognised standard for quantifying biofilm density. However, repeatability of data is difficult and provides very limited data concerning a complex system. To circumvent these issues, the inventors developed an agarose-based biofilm microcolony culture model designed to facilitate staining and visualisation of the colony to ascertain the presence of dispersed vs intact colonies, viability and biofilm density. This technique was used as a model to characterise the impact of D-Lactic acid treatment on biofilm microcolonies compared to tobramycin.

### Materials and Methods

### Biofilm Microcolony Culture

WACC91 and PA-001-11R (P. aeruginosa non-antibiotic resistant and resistant cultures) were thawed and streaked out on blood agar plates and allowed to grow at 37° C., 5% CO₂ for 12-24 hours until suitable growth was achieved to enable further culturing. An isolated colony was looped into 5 ml of LB broth and incubated overnight at 37° C., 5% CO₂.

Stock cultures were diluted 1:1000 in casting agarose made up in 1% of PMM to achieve approximately 10⁸ cfu/ml (confirmed by measuring 0.02 absorbance units, approximately 1.8 ul in 2 ml of agarose). Diluted working cultures were mixed well and seeded at 200 ul/well across a 96 well microtitre plate or per chamber of 8 chamber slide for confocal microscopy analysis. Biofilm formation was complete approximately 24-48 hours following seeding.

### Treatments

D-lactic acid and L-lactic acid were applied to antibiotic resistant and non-resistant P. aeruginosa bacterial biofilm colonies at 0.5-1.25 mg/ml independently or as a combined treatment for 24 hours. Tobramycin was applied to cultures at 16 ug/ml for 24 hours.

### Evaluation of Biofilm Disruption Using Confocal Microscopy

Media was removed from chamber slides and SYTO® 9 stain (0.5 ul/ml in PMM with arginine) with resazurin (0.0002% in PMM and arginine) added and allowed to incubate for 1 hr at 37° C. in 5% CO₂. Slides were then rinsed with PMM and fixed with 10% Neutral buffered formalin (NBF) overnight. Confocal was set to view resorufin at excitation 530/570 nm, emission 580-590 nm; and SYTO® 9 stain at excitation 483 nm, emission 503 nm.

### Results

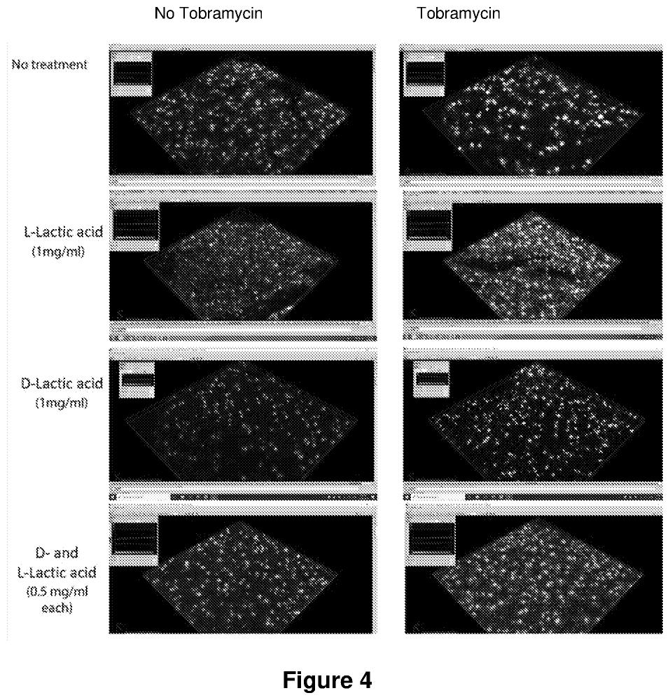

D-lactic acid was more effective than L-lactic acid in disruption of P. aeruginosa bacterial biofilms (FIG. 4). The treatment effect of tobramycin was enhanced when given with D-lactic acid. By combining D-lactic acid with antibiotics, D-lactic acid was shown to cause reversal of bacterial colony resistance to these antibiotics, in a user designed 3D biofilm model.

### Example 6

### D-Lactic Acid Prevents Biofilm Formation and Breaks Down Mature Biofilms

In order to validate the above results, experiments were conducted with Pseudomonas aeruginosa, Staphylococcus aureus and S. epidermidis using the crystal violet staining method for evaluating biofilms.

### Materials and Methods

### Biofilm Microcolony Culture

Cultures of interest were thawed and streaked out on LB agar or blood agar plates and allowed to grow at 37° C. for 12-24 hours until suitable growth was achieved to enable further culturing. An isolated colony was looped into 5 ml of LB broth and incubated overnight at 37° C. Stock cultures were then diluted 1:1000 in 1% of appropriate media (LB) to achieve approximately 10⁸ cfu/ml (confirmed by measuring 0.02 absorbance units, approximately 1.8 ul in 2 ml of media). Diluted working cultures were mixed well and seeded at 200 ul/well across a 96 well microtitre plate for biofilm treatment.

### Treatments

D-lactic acid, L-lactic acid, D/L lactic acid or glycolic acid was then added at 0.55 mM, 0.055 mM or 0.0055 mM (1×, 0.1× or 0.01×) final concentration in Phosphate Buffered Saline (final pH 7.0), either immediately after seeding to evaluate the effect on biofilm formation, or after 24 hours incubation at 37° C. to evaluate the effect on mature biofilms. After addition of treatments, all plates were then incubated for 24 hours at 37° C.

### Biofilm Disruption Evaluation

Biofilms were stained with 0.1% w/v of crystal violet (CV) in water to each well of the microtiter plate after the plate wells were cleared and washed by submersion in water (O'Toole 2011). Following incubation at room temperature for 15 minutes the microtiter plates were washed 3-4 times with water by submerging in water, with blotting to rid the plate of all excess cells and dye. Plates were left to dry overnight. The CV stain was solubilised with 125 ul of 30% acetic acid and absorbance to indicate biofilm biomass quantified at 550 nm following an incubation of 15 minutes, normalised to vehicle only.

### Results

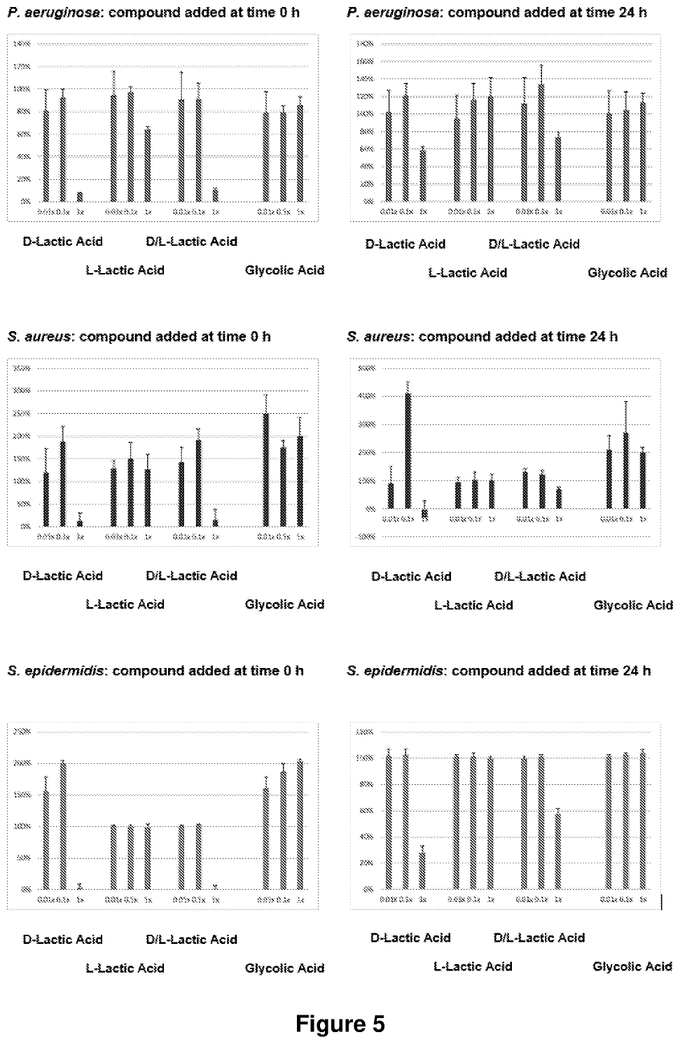

As shown in FIG. 5, a significant reduction in biofilm mass was seen for D-Lactic acid, but not for L-Lactic acid (compared to baseline) with P. aeruginosa, S. aureus and S. epidermidis. Effects were observed both for de novo forming biofilms (when D-Lactic acid or L-lactic acid were added at t=0) and for established biofilms (when D-Lactic acid or L-Lactic acid were added at t=24 hours). Note: 1×=0.55 mM for both lactic acid (D- or L-; 0.5 mg/mL) and glycolic acid.

The variable results the racemic mix (D/L) (depending on the timepoint added and the strain) is due the inefficiencies of the basic crystal violet assay. However, the results show the applicability of D-LA against biofilms produced by multiple pathogens.

The results for glycolic acid (2-hydroxy acetic acid), which is also known to have anti-bacterial properties but is achiral, further supports the proposal that the antibiofilm properties observed are dependent on the chirality of the molecule rather than any known antibacterial properties.

### Example 7

### D-Lactic Acid Causes Microcolony Dispersion

Biofilm disruption caused by D-lactic acid against Pseudomonas aeruginosa WACC91 was tested. The method used was that of Example 5 except that samples were fixed for staining/confocal at t=4 h after the addition of D- or L-LA.

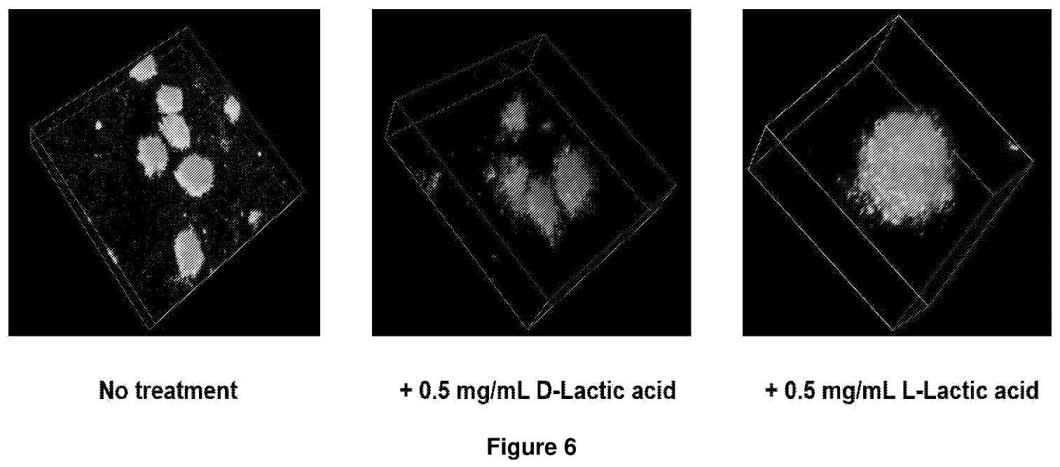

FIG. 6 shows that treatment with 1 mg/mL D-Lactic acid for 4 h caused biofilm colony dispersion and dispersion of individual bacteria, compared to the No Treatment control, or treatment with L-lactic acid.

### REFERENCES

Clinical and Laboratory Standards Institute. 2005. Performance standards for antimicrobial susceptibility testing; 15th informational supplement. CLSI/NCCLS M100-S15. Clinical and Laboratory Standards Institute, Wayne, Pa.
O'Toole, G. A. Microtiter Dish Biofilm Formation Assay. J. Vis. Exp. (47), e2437, doi: 10.3791/2437 (2011).
Kirchner, S., Fothergill, J. L., Wright, E. A., James, C. E., Mowat, E., Winstanley, C. Use of Artificial Sputum Medium to Test Antibiotic Efficacy Against Pseudomonas aeruginosa in Conditions More Relevant to the Cystic Fibrosis Lung. J. Vis. Exp. (64), e3857, doi: 10.3791/3857 (2012).

1. A composition for disrupting a biofilm, said composition comprising a 2-hydroxycarboxylic acid of the following formula:

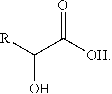

2. The composition of claim 1 wherein the 2-hydroxycarboxylic acid is a preferred enantiomer of a 2-hydroxycarboxylic acid with the following formula:

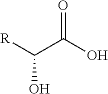

3. The composition of claim 2, further comprising an unpreferred enantiomer of a 2-hydroxycarboxylic acid with the following formula:

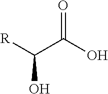

4. The composition of claim 3, wherein the % of unpreferred enantiomer of a 2-hydroxycarboxylic acid is less than 20%.

5. The composition of claim 2 wherein the composition does not comprise an unpreferred enantiomer of a 2-hydroxycarboxylic acid.

6. A method to disrupt a biofilm comprising the step of:
i. administering a 2-hydroxycarboxylic acid of any of claim 1 or 2 to the biofilm.

7. The method of claim 6 wherein the 2-hydroxycarboxylic acid is used in combination with an antimicrobial compound.

8. A composition comprising a 2-hydroxycarboxylic acid of any of claim 1 or 2 for treating or preventing a microbial infection in a subject, wherein the microbial infection is associated with a biofilm and wherein the composition disrupts the biofilm.

9. The composition of claim 8 wherein the composition further comprises an antimicrobial compound.

10. The use of a 2-hydroxycarboxylic acid of any of claim 1 or 2 in the manufacture of a composition for the disruption of a biofilm.

11. The use of a 2-hydroxycarboxylic acid of any of claim 1 or 2 for the disruption of a biofilm.

12. The use of a 2-hydroxycarboxylic acid of any of claim 1 or 2 in the manufacture of a composition for treating or preventing a microbial infection in a subject wherein the microbial infection is associated with a biofilm and wherein the composition disrupts the biofilm.

13. The use of a 2-hydroxycarboxylic acid of any of claim 1 or 2 for treating or preventing a microbial infection in a subject wherein the microbial infection is associated with a biofilm and wherein the composition disrupts the biofilm.

14. The use of a 2-hydroxycarboxylic acid of any of claim 1 or 2 for disrupting or preventing formation of a biofilm on a non-living surface.

15. The use of and of claims 10 to 14, wherein the 2-hydroxycarboxylic acid of any of claim 1 or 2 is used in conjunction with an antimicrobial compound.

16. The present invention provides a kit for the disruption of a biofilm, said kit comprising:
a) a 2-hydroxycarboxylic acid of any of claim 1 or 2; and
b) instructions for use.

17. The kit of claim 16 wherein the 2-hydroxycarboxylic acid is used in conjunction with an antimicrobial compound.

18. The composition of claim 1, wherein the 2-hydroxycarboxylic acid is D-lactic acid.
